(19) United States
(12) Patent Application Publication
(10) Pub. No.: US 2026/0290592 A1
(43) Pub. Date: Sep. 24, 2026

(54) SYSTEM

(71) Applicant: SoftBank Group Corp., Tokyo (JP)

(72) Inventor: Seiwa KOBAYASHI, Tokyo (JP)

(73) Assignee: SoftBank Group Corp., Tokyo (JP)

(21) Appl. No.: 19/564,712

(22) Filed: Mar. 12, 2026

(30) Foreign Application Priority Data
Mar. 19, 2025 (JP) 2025-044983

(51) Int. Cl. G16H 50/20

(52) U.S. Cl. CPC G16H 50/20

(57) ABSTRACT

A system includes a processor that is configured to provide, to a user terminal operated by a patient, an interface through which the patient inputs at least one of symptoms and treatment preferences in natural language, analyze information input via the interface by using a natural language processing technique to extract one or more keywords from the information, and generate a prompt that instructs a generative artificial intelligence model to generate additional information based on at least one of the patient's symptoms and the patient's treatment preferences, and input the prompt into the generative artificial intelligence model.

### CROSS-REFERENCE TO RELATED APPLICATION

This application is based on and claims priority under 35 USC 119 from Japanese Patent Application No. 2025-044983 filed on Mar. 19, 2025, the disclosure of which is incorporated by reference herein.

### BACKGROUND

### Technical Field

The present disclosure relates to a system.

### Related Art

Japanese Patent Application Laid-Open (JP-A) No. 2022-180282 discloses a persona chatbot control method executed by at least one processor. The method includes steps of: receiving a user utterance, adding the user utterance to a prompt including a description of a chatbot character and an associated instruction sentence, encoding the prompt, and inputting the encoded prompt to a language model to generate a chatbot utterance responding to the user utterance.

Conventional medical information systems and online health platforms largely rely on static content, simple search functions, or manually prepared decision trees to provide information and recommendations to patients. In the specific context of low back pain and other chronic conditions, patients frequently input their symptoms and treatment preferences in free-form natural language. However, existing systems often fail to accurately analyze such unstructured inputs, to capture key medical and preference-related concepts, and to connect them to dynamically generated, personalized information. As a result, patients receive generic or poorly targeted information that does not adequately reflect their individual symptoms, health status, or treatment preferences, which can reduce treatment adherence and overall treatment success rates.

Further, even when artificial intelligence is used, many systems do not provide a structured mechanism for generating prompts that effectively guide generative AI models. Without such structured prompts, generative AI output can be inconsistent, difficult to control, or misaligned with clinical goals, leading to information that is not optimally tailored to the patient's situation. In addition, conventional systems generally lack a feedback loop in which patient feedback regarding proposed treatment plans is used to iteratively refine such plans via generative AI, thereby limiting the potential for continuous improvement of treatment strategies.

Accordingly, there is a need for a system that can: (i) receive a patient's symptoms and treatment preferences in natural language; (ii) analyze the input using natural language processing to extract relevant keywords; (iii) generate structured prompts that instruct a generative AI model to produce additional information and customized treatment plans based on patient-specific data; and (iv) further generate prompts that enable the generative AI model to continuously improve the treatment plans in response to patient feedback, thereby supporting more accurate, adaptive, and effective treatment guidance and improving treatment success rates.

### SUMMARY

In order to solve the above-described problems, according to one aspect of the present invention, there is provided a system comprising a processor, wherein the processor is configured to provide, to a user terminal operated by a patient, an interface through which the patient inputs at least one of symptoms and treatment preferences in natural language, analyze information input via the interface by using a natural language processing technique to extract one or more keywords from the information, and generate a prompt that instructs a generative artificial intelligence model to generate additional information based on at least one of the patient's symptoms and the patient's treatment preferences, and input the prompt into the generative artificial intelligence model.

According to another aspect of the invention, the processor is further configured to generate, based on at least one of the patient's symptoms and the patient's health information, a prompt that instructs the generative artificial intelligence model to automatically generate a customized treatment plan for the patient, and input the prompt into the generative artificial intelligence model. By systematically structuring the prompt according to the extracted keywords, the patient's health status, and the patient's preferences, the system can cause the generative artificial intelligence model to output a detailed, patient-specific treatment plan that is more aligned with clinical objectives and the patient's lifestyle.

According to still another aspect of the invention, the processor is further configured to generate, based on patient feedback relating to a treatment plan obtained from the generative artificial intelligence model, a prompt that instructs the generative artificial intelligence model to continuously improve the treatment plan in order to increase a treatment success rate for the patient, and input the prompt into the generative artificial intelligence model. In this manner, the system creates an iterative feedback loop in which changes in pain level, adherence, or other patient-reported outcomes are converted into structured prompt content, and the generative artificial intelligence model is used to revise and optimize the treatment plan over time. By combining natural language processing of patient input, structured prompt generation, and iterative use of a generative artificial intelligence model, the invention enables provision of adaptive, high-quality, and personalized treatment information that addresses the shortcomings of conventional systems.

The term “system” refers to an integrated combination of hardware and software components, including at least one processor and associated memory, communication interfaces, and storage, configured to execute the functions described in the claims.

The term “processor” refers to one or more hardware processing units, such as a central processing unit (CPU), a graphics processing unit (GPU), a digital signal processor (DSP), or any combination thereof, capable of executing instructions to perform the functions described in the claims.

The term “user terminal” refers to any electronic device operated by a patient, such as a smartphone, tablet, personal computer, or dedicated medical device, that is capable of presenting an interface to the patient and communicating with the system over a network.

The term “interface” refers to a graphical user interface, voice-based interface, or other input/output mechanism provided to the user terminal that allows a patient to input information, including symptoms and treatment preferences, and to receive information from the system.

The term “patient” refers to a human user who suffers from one or more health-related conditions, such as symptoms of a disease or disorder, and who uses the system to obtain information or treatment guidance.

The term “symptoms” refers to patient-reported manifestations of a health condition, including but not limited to descriptions of pain, discomfort, functional limitations, duration of such manifestations, and situational triggers.

The term “treatment preferences” refers to the patient's stated wishes or constraints regarding treatment modalities, including but not limited to preferences for or against exercise therapy, medication, surgery, manual therapy, or other intervention types.

The term “health information” refers to data describing the patient's health status and background, including but not limited to age, weight, medical history, comorbidities, current medications, activity level, and prior treatments.

The term “natural language” refers to human languages such as English, Japanese, or other spoken or written languages, as opposed to formal programming languages or fixed-form structured input formats.

The term “natural language processing technique” refers to any computational method or algorithm for analyzing and processing natural language text, including but not limited to tokenization, part-of-speech tagging, named entity recognition, syntactic or semantic parsing, text classification, and keyword extraction.

The term “keyword” refers to a word, phrase, or symbol extracted from a natural language input that represents a medically or contextually relevant concept, such as a particular symptom, body part, severity, treatment modality, or temporal descriptor.

The term “generative artificial intelligence model” refers to a machine learning model configured to generate text or other content in response to an input, such as a large language model or other generative model trained on data, which can output additional information or treatment plans based on prompts.

The term “prompt” refers to an input instruction, including text and optionally structured data, provided to the generative artificial intelligence model to define a task, constrain output content, and specify conditions under which the model is to generate additional information or a treatment plan.

The term “additional information” refers to information generated by the generative artificial intelligence model in response to a prompt, other than the original patient input, including but not limited to explanatory text, educational content, recommendations, or clarifying questions tailored to the patient's symptoms and preferences.

The term “treatment plan” refers to a structured or semi-structured set of recommended medical or healthcare-related actions for the patient, which may include exercise regimens, lifestyle modifications, therapy schedules, or other instructions intended to address the patient's symptoms or condition.

The term “customized treatment plan” refers to a treatment plan that is automatically generated by the generative artificial intelligence model based on patient-specific data, including at least one of the patient's symptoms and the patient's health information, and that is tailored to the individual characteristics and needs of the patient.

The term “patient feedback” refers to information provided by the patient regarding the execution or outcome of a treatment plan, including but not limited to reported changes in symptoms, pain levels, adherence to the plan, side effects, satisfaction, or qualitative comments about the plan.

The term “continuously improve” refers to revising or updating a treatment plan one or more times over a treatment period based on newly obtained patient feedback or additional patient data, so that the treatment plan becomes progressively better aligned with the patient's condition and treatment goals.

The term “treatment success rate” refers to a measure or likelihood of achieving desired clinical or patient-centered outcomes, such as reduction of symptoms, improved function, higher patient satisfaction, or achievement of predefined therapeutic goals, when a treatment plan is applied.

### BRIEF DESCRIPTION OF THE DRAWINGS

Exemplary embodiments of the present disclosure will be described in detail based on the following figures, wherein:

FIG. 1 is a schematic diagram illustrating an example of a configuration of a data processing system according to a first exemplary embodiment;

FIG. 2 is a schematic diagram illustrating an example of relevant functions of a data processing device and a smart device according to the first exemplary embodiment;

FIG. 3 is a schematic diagram illustrating an example of a configuration of a data processing system according to a second exemplary embodiment;

FIG. 4 is a schematic diagram illustrating an example of relevant functions of a data processing device and smart glasses according to the second exemplary embodiment;

FIG. 5 is a schematic diagram illustrating an example of a configuration of a data processing system according to a third exemplary embodiment;

FIG. 6 is a schematic diagram illustrating an example of relevant functions of a data processing device and a headset-type terminal according to the third exemplary embodiment;

FIG. 7 is a schematic diagram illustrating an example of a configuration of a data processing system according to a fourth exemplary embodiment;

FIG. 8 is a schematic diagram illustrating an example of relevant functions of a data processing device and a robot according to the fourth exemplary embodiment;

FIG. 9 illustrates an emotion map mapping plural emotions;

FIG. 10 illustrates an emotion map mapping plural emotions;

FIG. 11 is a sequence diagram showing the flow of data processing system processing in Example 1;

FIG. 12 is a sequence diagram showing the flow of data processing system processing in Application Example 1;

FIG. 13 is a sequence diagram showing the flow of data processing system processing in Example 2; and

FIG. 14 is a sequence diagram showing the flow of data processing system processing in Application Example 2.

### DETAILED DESCRIPTION

Description follows regarding an example of exemplary embodiments of a system according to technology disclosed herein, with reference to the appended drawings.

First, explanation follows regarding terminology employed in the following description.

In the following exemplary embodiments, a reference-numeral-appended processor (hereinafter simply referred to as “processor”) may be implemented by a single computation unit, and may be implemented by a combination of plural computation units. The processor may be implemented by a single type of computation unit, or may be implemented by a combination of plural types of computation units. Examples of computation unit include a central processing unit (CPU), a graphics processing unit (GPU), a general-purpose computing on graphics processing units (GPGPU), an accelerated processing unit (APU), and the like.

In the following exemplary embodiments, random access memory (RAM) appended with a reference numeral is memory temporarily stored with information, and is employed as working memory by a processor.

In the following exemplary embodiments, reference-numeral-appended storage is a single or plural non-volatile storage devices for storing various programs and various parameters and the like. Examples of non-volatile storage devices include flash memory (such as a solid state drive (SSD)), a magnetic disk (for example, a hard disk), magnetic tape, and the like.

In the following exemplary embodiments, a reference-numeral-appended communication interface (I/F) is an interface including a communication processor and an antenna or the like. The communication I/F has the role of communicating between plural computers. An example of a communication standard applied for the communication I/F is a wireless communication standard, such as a Fifth Generation Mobile Communication System (5G), Wi-Fi (registered trademark), Bluetooth (registered trademark), and the like.

In the following exemplary embodiments “A and/or B” has the same definition as “at least one out of A or B”. Namely, “A and/or B” may mean A alone, may mean B alone, or may mean a combination of A and B. Moreover, similar logic to “A and/or B” is applied when “and/or” is employed to link three or more items in the present specification.

### First Exemplary Embodiment

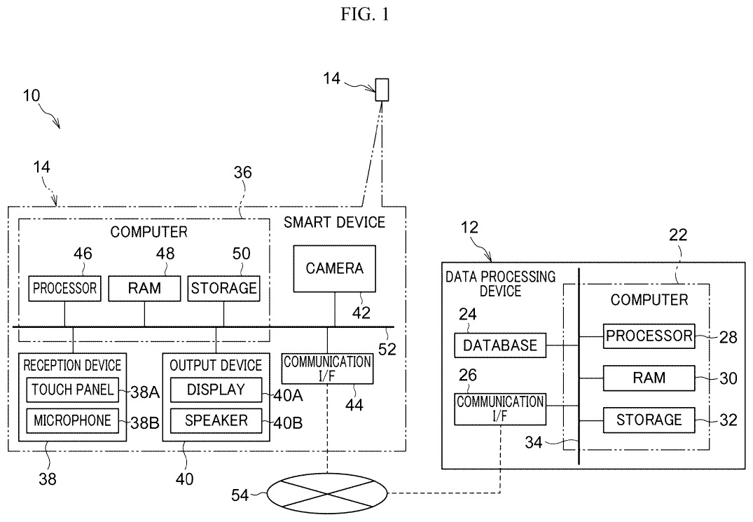

FIG. 1 illustrates an example of a configuration of a data processing system 10 according to a first exemplary embodiment.

As illustrated in FIG. 1, the data processing system 10 includes a data processing device 12 and a smart device 14. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart device 14 includes a computer 36, a reception device 38, an output device 40, a camera 42, and a communication I/F 44. The computer 36 includes a processor 46, RAM 48, and storage 50. The processor 46, the RAM 48, and the storage 50 are connected to a bus 52. The reception device 38, the output device 40, the camera 42, and the communication I/F 44 are also connected to the bus 52.

The reception device 38 includes a touch panel 38A, a microphone 38B, and the like for receiving user input. The touch panel 38A receives user input from contact of a pointer (for example, a pen, a finger, or the like) by detecting contact of the pointer. The microphone 38B receives spoken user input by detecting speech of the user. A control unit 46A in the processor 46 transmits data representing the user input received by the touch panel 38A and the microphone 38B to the data processing device 12. A specific processing unit 290 in the data processing device 12 acquires the data indicating the user input.

The output device 40 includes a display 40A, a speaker 40B, and the like for presenting data to a user 20 by outputting the data in an expression format perceivable by the user 20 (for example, audio and/or text). The display 40A displays visual information such as text, images, or the like under instruction from the processor 46. The speaker 40B outputs audio under instruction from the processor 46. The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like.

The communication I/F 44 is connected to the network 54. The communication I/F 44 and the communication I/F 26 perform the role of exchanging various information between the processor 46 and the processor 28 over the network 54.

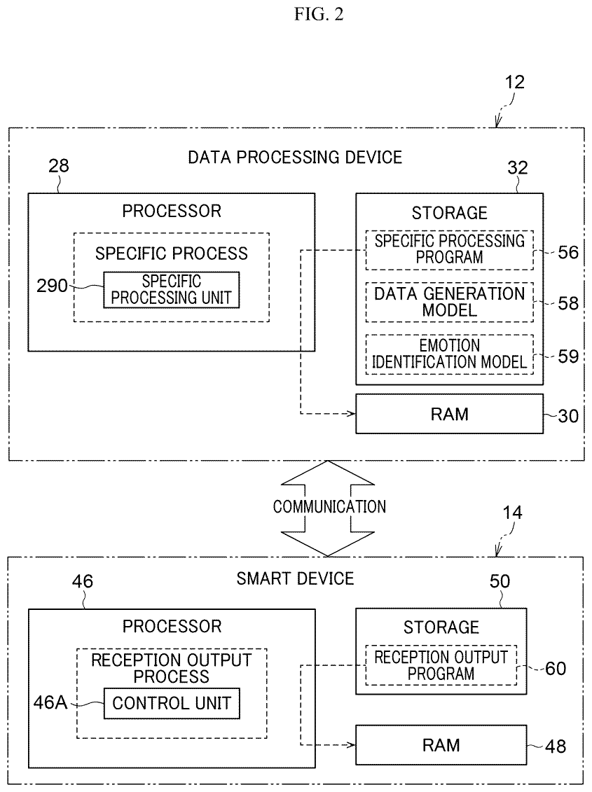

FIG. 2 illustrates an example of relevant functions of the data processing device 12 and the smart device 14.

As illustrated in FIG. 2, specific processing is performed by the processor 28 in the data processing device 12. A specific processing program 56 is stored in the storage 32. The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

A data generation model 58 and an emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart device 14. A reception and output program 60 is stored in the storage 50. The reception and output program 60 is employed by the data processing system 10 in combination with the specific processing program 56. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48. Note that a configuration may be adopted in which a similar data generation model and emotion identification model to the data generation model 58 and the emotion identification model 59 are included in the smart device 14, and these models are used to perform similar processing to the specific processing unit 290. The reception and output program is implemented by the processor 46 operating as the control unit 46A according to the reception and output program 60 executed in the RAM 48.

Note that devices other than the data processing device 12 may include the data generation model 58. For example, a server device (for example, a generation server) may include the data generation model 58. In such cases, the data processing device 12 performs communication with the server device including the data generation model 58 to obtain a processing result (prediction result or the like) obtained using the data generation model 58. The data processing device 12 may be a server device, and may be a terminal device owned by the user (for example, a mobile phone, a robot, a home electrical appliance, or the like). Next, description follows regarding an example of processing by the data processing system 10 according to the first exemplary embodiment.

### Example 1

Description follows regarding a flow of the specific processing in an Example 1. The units of the system described below are implemented by the data processing device 12 and the smart device 14. The data processing device 12 is called a “server” and the smart device 14 is called a “terminal”.

Conventional medical information systems that support patients in finding healthcare service points, such as clinics or treatment centers, typically rely on rigid form-based search conditions and simple keyword matching mechanisms implemented in databases or basic search engines. These systems require users to manually adapt their symptom descriptions and treatment preferences to predefined fields or controlled vocabularies. As a result, such systems often fail to correctly interpret free-form natural language input from users, especially when users describe complex combinations of symptoms, subjective preferences, and contextual constraints. This leads to low-quality retrieval results, increased user interaction cycles, and a high cognitive burden on users who must iteratively refine queries to obtain meaningful recommendations.

Furthermore, existing systems generally treat database search and intelligent recommendation as separate, loosely coupled processes. Database engines may return a broad list of candidate healthcare service points based on coarse conditions, while any recommendation or explanation is either static content or manually curated text. There is no tightly integrated computational mechanism that combines structured search results with dynamically generated, context-aware explanations, rankings, and refinements produced by advanced machine learning models. This separation results in suboptimal use of available computational resources, redundant processing, and the inability to adapt recommendations to nuanced patient conditions and evolving feedback.

In addition, many systems that attempt to incorporate machine learning models do not control these models via explicit, structured prompts that reflect both analyzed natural language input and structured facility data. Instead, they use generic model queries that do not leverage the full richness of user-specific and context-specific information already available in the system. Consequently, the generative output may be misaligned with backend data, inconsistent with actual healthcare service point capabilities, or difficult to integrate back into the system's data structures, leading to unreliable or opaque recommendations.

Moreover, conventional systems offer only limited support for iterative improvement of individualized treatment plans. Even when an initial plan is generated, there is often no computational loop that formally incorporates user evaluation information and longitudinal progress information into the model's input in a structured way. As a result, the system cannot systematically update treatment plans based on objective and subjective feedback, and must rely on ad hoc manual adjustments by healthcare professionals or the users themselves. This reduces the effectiveness and adaptability of the system and fails to exploit the capabilities of modern generative models to synthesize and revise complex treatment strategies.

From a computer-technology perspective, there is a need for an improved data processing architecture that (i) systematically transforms unstructured natural language inputs into abstracted, machine-usable representations, (ii) coordinates database search and search-engine retrieval using those representations, (iii) formulates precise prompt sentences for a generative model that reflect both user input and retrieved structured data, and (iv) programmatically integrates the generative output back into the system in a deterministic and verifiable manner. There is also a need to support an iterative refinement loop in which user feedback and time-series progress information are encoded and supplied to the generative model to compute updated, improved treatment plans. Without such a coordinated architecture, systems are unable to provide consistent, high-quality, and explainable recommendations, and cannot reliably scale to diverse users and medical conditions. Accordingly, the technical problem to be solved is to improve the functioning of computer-based medical information systems by providing a unified processor-controlled workflow that: (1) accurately analyzes and abstracts natural language user input, (2) efficiently retrieves and structures candidate healthcare service point information, (3) generates and applies precise prompt sentences to control a generative information processing apparatus, and (4) computationally integrates and iteratively updates recommendations and individualized treatment plans in a way that reduces user interaction burden, improves recommendation quality, and enhances the overall performance and reliability of the system as a data processing platform.

The specific processing by the specific processing unit 290 of the data processing device 12 in Example 1 is realized by the following means.

The present invention provides a server comprising a processor configured to provide an information input and output interface through which a user inputs physical condition information and preference condition information, to obtain natural language data transmitted from a use terminal and to extract, by language analysis processing including tokenization, part-of-speech tagging, and important term extraction, abstract matters related to symptoms and abstract matters related to preference conditions from the natural language data, to set a search condition for an information storage device that stores healthcare service point information based on the extracted abstract matters and to execute a search process including structured query processing and full-text search to acquire candidate healthcare service point information, to generate a prompt sentence to be input to a generative information processing apparatus based on the extracted abstract matters and the acquired healthcare service point information, to input the prompt sentence to the generative information processing apparatus and to obtain, from the generative information processing apparatus, generated information including a selection result of healthcare service points suitable for the user's symptoms and preference conditions and explanatory information related to the selection result, to compare and associate the acquired healthcare service point information with the generated information and to generate integrated healthcare service point information in which ranking, filtering, and addition of explanatory information of the healthcare service points are performed based on the generated information, and to transmit the integrated healthcare service point information to the use terminal and to provide output data in a displayable format to the user, and further configured, in at least some embodiments, to construct the prompt sentence so as to instruct automatic generation of an individualized treatment plan including time-series treatment content and lifestyle guidance content and to iteratively update the individualized treatment plan based on evaluation information and progress information from the user obtained via the interface. This enables an improved computer-implemented medical information system that more accurately interprets natural language input, more efficiently retrieves and structures healthcare service point information, more effectively controls a generative information processing apparatus by structured prompt sentences, and more reliably generates and updates integrated recommendations and individualized treatment plans, thereby enhancing the technical performance, responsiveness, and reliability of the overall data processing architecture.

The term “user” refers to an individual who operates a use terminal to input physical condition information and preference condition information and to receive output data from the system.

The term “use terminal” refers to an electronic apparatus, such as a mobile device, a personal computer, or a similar computing device, that executes a user interface and communicates natural language data and displayable output data with the server over a communication network.

The term “server” refers to an electronic apparatus or group of electronic apparatuses that includes at least one processor and at least one memory, executes programs to perform the data processing described herein, and communicates with one or more use terminals via a communication network.

The term “processor” refers to a hardware processing unit, such as a central processing unit or an execution core thereof, or a combination of such units, that executes machine-readable instructions to implement the functions of the server described in this specification.

The term “memory” refers to a hardware storage element, such as a semiconductor memory, a magnetic storage device, or an optical storage device, that stores programs and data used by the processor.

The term “information input and output interface” refers to a combination of software and hardware functions through which the server causes the use terminal to display input screens and output screens, and through which the server receives user input and transmits displayable output data.

The term “physical condition information” refers to information describing a state of a body of the user, including, for example, symptoms, duration, severity, and medical history, expressed as natural language or structured data.

The term “preference condition information” refers to information indicating a desire or constraint of the user regarding treatment or healthcare services, including, for example, preferred treatment methods, time constraints, location constraints, and cost constraints.

The term “natural language data” refers to character string data that represents human language expressions, such as sentences or phrases input by the user, without requiring adherence to predefined structured formats or controlled vocabularies.

The term “language analysis processing” refers to processing operations executed by the processor on natural language data, including at least tokenization, part-of-speech tagging, and important term extraction, to convert the natural language data into an internal representation suitable for further computation.

The term “tokenization” refers to a processing operation that segments natural language data into units, such as words, subwords, or phrases, for subsequent analysis.

The term “part-of-speech tagging” refers to a processing operation that assigns grammatical category labels, such as noun, verb, or adjective, to tokens obtained by tokenization.

The term “important term extraction” refers to a processing operation that selects, from among tokens or groups of tokens, terms that are determined to be relevant to a specified purpose, such as identifying symptoms or preferences from natural language data.

The term “abstract matters related to symptoms” refers to conceptual representations derived from natural language data that characterize medical conditions, such as symptom categories, affected body parts, duration, or severity, independent of specific wording used by the user.

The term “abstract matters related to preference conditions” refers to conceptual representations derived from natural language data that characterize user preferences or constraints, such as preferred treatment types, scheduling requirements, or geographical constraints, independent of specific wording used by the user.

The term “information storage device” refers to a hardware storage system, including one or more storage media and a control unit, that stores healthcare service point information and responds to search requests issued by the processor.

The term “healthcare service point information” refers to information about facilities or entities that provide healthcare-related services, including, for example, identifiers, names, locations, available treatments, specialties, and operational conditions.

The term “search condition” refers to parameter data generated by the processor, based on abstract matters, that specifies criteria for selecting records from the information storage device.

The term “structured query processing” refers to a search operation in which the processor issues a query expressed in a structured query language or equivalent formalism to retrieve records from the information storage device based on defined fields and conditions.

The term “full-text search” refers to a search operation that analyzes textual content stored in the information storage device to find records whose text fields match or are similar to input terms according to a defined scoring or matching algorithm.

The term “candidate healthcare service point information” refers to a subset of healthcare service point information retrieved from the information storage device that satisfies at least part of the search condition.

The term “generative information processing apparatus” refers to an information processing system, such as a server or service executing a generative AI model, that generates output information in response to input data, including prompt sentences, by performing probabilistic or statistical inference.

The term “generative AI model” refers to a machine learning model, such as a neural network-based language model, that generates text or other data formats in response to input sequences, using learned parameters obtained from training on a dataset.

The term “prompt sentence” refers to input data, typically expressed as text, generated by the processor to instruct the generative information processing apparatus regarding a content or style of output information to be generated.

The term “generated information” refers to output data produced by the generative information processing apparatus in response to a prompt sentence, including, for example, recommended healthcare service points, explanatory text, or treatment plans.

The term “selection result of healthcare service points” refers to generated information that identifies one or more healthcare service points determined by the generative information processing apparatus to be suitable for given symptoms and preference conditions.

The term “explanatory information” refers to generated information that describes reasons, context, or additional details regarding a selection result or recommendation produced by the generative information processing apparatus.

The term “integrated healthcare service point information” refers to data generated by the processor by combining candidate healthcare service point information retrieved from the information storage device with generated information obtained from the generative information processing apparatus.

The term “ranking” refers to a processing operation that assigns an order or score to healthcare service points based on one or more criteria, including generated information, to indicate relative suitability or priority.

The term “filtering” refers to a processing operation that removes or excludes some healthcare service points from a set of candidates based on specified criteria, including generated information or user constraints.

The term “addition of explanatory information” refers to a processing operation that associates explanatory information, obtained from the generative information processing apparatus, with corresponding healthcare service points in integrated healthcare service point information.

The term “output data in a displayable format” refers to data structured so that a use terminal can present the data to a user via a user interface, such as text, lists, or graphical elements.

The term “individualized treatment plan” refers to a treatment-related schedule or set of recommendations generated for a specific user based on physical condition information, preference condition information, and healthcare service point information.

The term “time-series treatment content” refers to treatment-related actions, sessions, or procedures arranged along a time axis, such as daily, weekly, or monthly treatment steps specified for a user.

The term “lifestyle guidance content” refers to recommendations related to daily habits, exercises, or environmental adjustments intended to support or complement medical treatments for a user.

The term “evaluation information” refers to data indicating the user's subjective or objective assessment of an individualized treatment plan or of received healthcare services, including satisfaction levels, perceived effectiveness, or reported side effects.

The term “progress information” refers to data representing a temporal change in the user's physical condition or treatment status, including symptom changes, adherence information, or outcome measurements over time.

The term “improvement proposal” refers to generated information produced by the generative information processing apparatus that suggests modifications to an existing individualized treatment plan based on evaluation information and progress information.

In one embodiment, a server includes at least one processor, at least one memory, a network interface, and non-transitory storage that stores executable programs and data structures. The server executes an operating system such as a general-purpose server operating system and executes an application program that implements the functions described below. The server communicates with one or more terminals via a communication network, such as the Internet or a wireless mobile network.

A terminal includes a processing unit, a memory, a display device, an input device such as a touch panel or keyboard, and a communication interface. The terminal executes a web browser or a native application. The terminal displays user interfaces generated by the server and transmits natural language input and control commands to the server.

A user operates the terminal to access an information input and output interface provided by the server. The user inputs physical condition information and preference condition information as free-text sentences, such as “I have chronic low back pain for more than 6 months and I prefer exercise therapy rather than medication” or “I want a clinic near my workplace that offers evening sessions.” The terminal transmits this natural language data to the server in a structured message format, such as a character string encoded in a request payload.

The server stores the received natural language data in a memory-resident data structure, for example as a record containing at least a user identifier, a timestamp, and two text fields for physical condition information and preference condition information. The server applies language analysis processing to this natural language data by executing a natural language processing library such as a general-purpose toolkit, in a scripting language environment such as a Python environment running on the processor.

The server uses tokenization functions to segment the input text into tokens. The server uses part-of-speech tagging functions to assign grammatical categories to each token. The server optionally uses named entity recognition to identify terms corresponding to medical conditions, anatomical regions, or therapeutic modalities. The server uses important term extraction logic that performs stop-word removal, stemming or lemmatization, and domain-specific filtering, to produce a compact representation of symptoms and preferences. For example, the server derives abstract matters such as “low back region,” “chronic duration,” “exercise therapy,” and “evening availability.”

The server normalizes these abstract matters into an internal representation, such as a vector or a set of key-value pairs, that is used to access structured data in an information storage device. The server stores healthcare service point information in a relational database management system, such as a SQL-based database, and in a search engine index, such as an inverted index provided by a search engine platform. The server uses structured query processing to retrieve records from the relational database based on normalized fields, such as symptoms treated, therapies provided, and location. The server uses full-text search via the search engine to retrieve records based on description fields and unstructured attributes.

The server applies a non-trivial combination of structured query processing and full-text search. For example, the server uses structured query processing to restrict results to facilities treating spinal conditions and then applies full-text search over unstructured facility descriptions to rank facilities that emphasize “exercise-based rehabilitation for chronic low back pain.” The server stores the retrieved candidate healthcare service point information in an internal data structure such as a list of objects, where each object includes a facility identifier, a location vector, a set of treatment tags, and descriptive text.

The server generates a prompt sentence for a generative AI model, which is implemented as a generative information processing apparatus. In one embodiment, the generative AI model is a neural network-based language model of the transformer architecture, having multiple self-attention layers, feed-forward layers, layer normalization, and learned embeddings for tokens and positional indices. The generative AI model has been trained on a mixture of general text data and domain-specific medical-related text data. During training, the model uses an auto-regressive objective with a cross-entropy loss function and gradient-based optimization, such as stochastic gradient descent with adaptive moment estimation. The model updates parameters, such as weight matrices in attention layers and feed-forward layers, by backpropagating gradients computed from the loss function. The server does not train the model in the production system; instead, the server uses a pre-trained model and supplies inference requests.

The server constructs the prompt sentence so that it explicitly encodes the abstract matters and the candidate healthcare service point information into a linear token sequence. The server structures the prompt sentence to provide the generative AI model with both contextual information and explicit instructions. For example, the server can generate a prompt sentence such as:

“A patient reports chronic low back pain that has persisted for more than 6 months and prefers exercise therapy instead of medication. The patient wants a healthcare facility near their workplace and is available on weekday evenings. Given the following list of healthcare facilities and their specialties, select the most suitable facilities and explain in simple language why each facility is appropriate for this patient.”

The server then appends a textual enumeration of facility names, locations, and treatment tags, expressed as sentences. As another example, the server can generate a prompt sentence such as:

“For a patient with chronic low back pain who wants to avoid surgery and medication and is interested in exercise therapy, summarize a three-month treatment schedule that includes specific exercise sessions and lifestyle guidance, using the available facilities that provide supervised exercise programs.”

The server adjusts the content and format of the prompt sentence according to the type of generated information required, such as facility selection, explanatory text, or individualized treatment plans.

The server transmits the prompt sentence to the generative information processing apparatus via an application programming interface. The generative information processing apparatus executes the generative AI model on specialized hardware, for example using multiple graphics processing units and high-bandwidth memory. The generative AI model receives the prompt sentence as a sequence of tokens, transforms each token into an embedding vector, and applies successive transformer layers, each applying multi-head self-attention and non-linear transformations. The model computes a probability distribution over the vocabulary for each output position and selects tokens based on this distribution, for example using nucleus sampling or greedy decoding, to generate an output sequence representing generated information.

The server receives the generated information from the generative information processing apparatus. The server parses the generated information, which may be structured as text segments delineating facility names, reasons, and treatment steps. The server uses rule-based parsing and pattern matching to align facility names in the generated information with facility identifiers in the candidate healthcare service point information. The server then computes a ranking for each facility based on both search engine scores and generated statements. For example, the server may assign a higher score to facilities that are referenced in the generated information with positive explanatory statements and penalize facilities that are not referenced.

The server generates integrated healthcare service point information by merging the candidate healthcare service point information and the generated information. The server attaches explanatory information generated by the generative AI model, such as “This facility focuses on exercise-based rehabilitation and offers evening sessions suitable for working patients with chronic low back pain,” to each corresponding facility record. The server filters out facilities that the model or rules indicate as less suitable, thereby reducing the list to a manageable number of facilities. The server stores the integrated healthcare service point information in a structured format in memory.

The server transmits the integrated healthcare service point information to the terminal. The terminal renders a list of facilities with attached explanations on the display device. The user can select a particular facility to view detailed information. The terminal displays attributes such as location, treatment types, expected treatment schedule, and generated explanatory comments. The user can provide evaluation information and progress information through the same interface, for example by rating symptom relief, entering free-text comments about side effects, and recording attendance at sessions.

The server receives evaluation information and progress information from the terminal and stores them in a structured profile for the user. The server incorporates this information into a new prompt sentence for the generative AI model. For example, the server can generate a prompt sentence such as:

“The patient with chronic low back pain has followed the recommended exercise therapy plan for 4 weeks. The patient reports moderate improvement in pain but difficulty performing certain exercises due to knee discomfort. Given the original treatment plan and the following notes about progress, please propose modifications to the treatment plan that reduce strain on the knees while maintaining effectiveness for low back pain.”

The server supplies this prompt sentence to the generative AI model, obtains an improvement proposal, and updates the individualized treatment plan accordingly. The server then associates the updated individualized treatment plan with the integrated healthcare service point information and transmits the updated plan to the terminal.

This architecture provides technical effects that go beyond mere automation of human decision making. The server uses specific data structures for abstract matters, search conditions, and integrated healthcare service point information, which allow efficient indexing, caching, and retrieval operations. The combination of structured query processing and full-text search, driven by abstract matters extracted by language analysis, reduces the size of candidate sets and improves recall and precision compared to naïve keyword search. The explicit construction of prompt sentences that integrate both abstract matters and structured facility data allows the generative AI model to perform constrained generation aligned with backend data. This reduces hallucinated information and improves the consistency and reliability of the generated information.

By pre-processing user input into abstract matters and by encoding candidate facility attributes into structured segments within the prompt sentence, the server reduces the token length needed to represent relevant information, thereby lowering network transmission volume to the generative information processing apparatus and reducing inference time. The server also reuses intermediate representations, such as normalized symptom vectors and facility embeddings, across multiple requests, which improves throughput and reduces computational redundancy.

The generative AI model architecture contributes to technical improvement because the transformer-based model, trained with domain-adapted data and loss functions, can represent complex relationships among symptoms, treatments, and facility attributes that cannot be easily encoded in traditional rule-based systems. The server uses the model as a function approximator that computes mappings from combined text and structured data representations to probability distributions over possible recommendations. The specific training process, including the selection of training data, the use of cross-entropy loss, and gradient-based updates of high-dimensional weight matrices, yields a model that can generalize to new combinations of symptoms and preferences, thereby improving coverage and accuracy compared to static rule sets.

The system improves computing performance by distributing tasks between the relational database, the search engine, and the generative AI model. The database handles structured predicates, the search engine handles free-text similarity, and the generative AI model handles context-sensitive reasoning and text generation. This division of labor, combined with caching of intermediate representations and ranking scores, reduces overall processing time for each user request and allows the server to handle more concurrent users. The use of abstract matters also reduces the complexity of queries transmitted to the database and search engine, improving index utilization and lowering read latency.

In further embodiments, the server can execute alternative language analysis methods, such as using a smaller, locally hosted neural sequence model to pre-encode user input into semantic vectors, which are then used both for database retrieval and as part of the prompt sentence construction. The server can also employ different generative AI models, such as encoder-decoder architectures, or models fine-tuned specifically for medical recommendation tasks. The server can vary decoding strategies, such as using constrained decoding to prevent the model from generating facility identifiers not present in the candidate list.

In another embodiment, the terminal can perform part of the language analysis, such as preliminary tokenization, to reduce latency and server load. The server then receives pre-processed tokens and applies higher-level processing. In yet another embodiment, the server can maintain user-specific preference profiles that are learned over time using statistical models, and inject these profiles as additional context into the prompt sentences.

By defining specific data structures, processing flows, and model interaction protocols, the system achieves reduced error rates in matching facilities to user needs, more stable treatment plan generation, and faster response times, thereby improving the technical functioning of the computer-based medical information system as a whole.

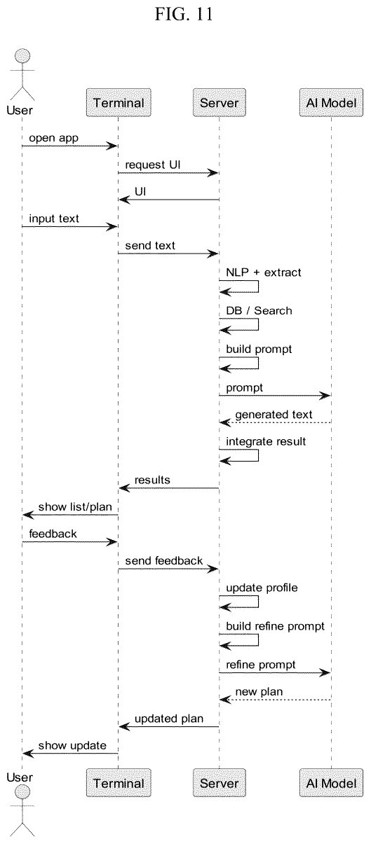

The following describes the processing flow using FIG. 11.

### Step 1:

User operates the terminal to access the system's input interface.

User opens a web page or a native application on the terminal and selects a function for searching healthcare service points or for generating a treatment plan.

Input: No prior data; only a request to open the interface.

Output: A request message from the terminal to the server to obtain an input screen.

### Step 2:

Server receives the request and generates an input screen.

Server executes an application program, constructs an HTML form or application view containing text fields for physical condition information and preference condition information, and sends the screen definition to the terminal.

Input: Request message from the terminal indicating the type of interface needed.

Output: Screen data (HTML, CSS, UI layout data) transmitted to the terminal.

### Step 3:

Terminal renders the input screen and accepts user text input.

Terminal executes a browser or application runtime, draws text boxes, labels, and buttons on the display, and waits for user operations. User types symptom descriptions and preference conditions, such as “I have chronic low back pain for 6 months and prefer exercise therapy” and “I need evening appointments near my office,” using a keyboard or touch panel.

Input: Screen data from the server and user keystrokes or touch events.

Output: Natural language text strings held in input fields within the terminal.

### Step 4:

Terminal validates the input and transmits natural language data to the server.

Terminal executes validation logic to check that required fields are not empty and that text length is within permissible limits. Terminal then packs the text into a structured message and sends it to the server using a network protocol.

Input: Natural language text entered by the user in the input fields.

Output: A request payload containing physical condition text and preference condition text transmitted to the server.

### Step 5:

Server receives the natural language data and stores it in internal data structures.

Server parses the request payload, extracts the text fields, and stores them together with a user identifier and timestamp in memory and optionally in a log table. Server forms a combined text string, for example by concatenating the physical condition text and preference condition text.

Input: Request payload containing user-entered natural language text.

Output: Structured in-memory records containing combined text and associated metadata.

### Step 6:

Server performs language analysis processing on the combined text.

Server calls a natural language processing library to tokenize the text into tokens, assign part-of-speech tags, and identify key terms. Server removes stop-words and applies normalization, such as lowercasing and stemming or lemmatization. Server then groups tokens into phrases corresponding to symptoms and preferences.

Input: Combined natural language text string and tokenization/tagging models stored in memory.

Output: A list of normalized tokens and phrases with associated grammatical and semantic tags.

### Step 7:

Server derives abstract matters related to symptoms and preference conditions.

Server applies domain-specific mapping rules to convert tokens and phrases into abstract representations, such as symptom categories, anatomical regions, duration attributes, and treatment preference attributes. For example, server maps “chronic low back pain for 6 months” to a symptom category “chronic lumbar pain” with duration “>3 months,” and maps “prefer exercise therapy” to a treatment preference “exercise-based, non-pharmacological.”

Input: Tokenized and tagged phrases from the language analysis processing.

Output: Abstract matter objects representing symptom attributes and preference attributes.

### Step 8:

Server constructs search conditions for the information storage device.

Server converts abstract matters into structured query parameters, such as fields for symptom category, treatment type, time availability, and location constraints. Server forms a structured query for a relational database and a text query for a search engine index.

Input: Abstract matter objects derived from the natural language data.

Output: Structured query definition (for example, SQL-like predicates) and full-text search query specification.

### Step 9:

Server executes structured query processing on the healthcare information database.

Server sends the structured query to a relational database that stores healthcare service point records. The database performs index lookups and filtering based on fields such as specialty, available therapies, and operating hours, and returns records that satisfy the query conditions.

Input: Structured query parameters and the relational database content.

Output: A set of candidate healthcare service point records retrieved from the database.

### Step 10:

Server executes full-text search on a search engine index.

Server sends the text query, composed of key terms and phrases, to a search engine that maintains an inverted index over textual fields such as facility descriptions and treatment explanations. The search engine computes similarity scores, ranks documents, and returns a list of facility identifiers with relevance scores.

Input: Full-text search query and indexed textual data.

Output: Ranked search engine results containing facility identifiers and relevance scores.

### Step 11:

Server merges and normalizes candidate healthcare service point information.

Server joins database records from structured query processing with records referenced in the search engine results by matching facility identifiers. Server calculates a combined score for each facility by applying a weighting function to structured query matches and full-text relevance scores. Server creates a normalized candidate list with identifiers, location data, treatment tags, and descriptive text.

Input: Candidate records from the database and ranked results from the search engine.

Output: A unified list of candidate healthcare service point objects with combined scores.

### Step 12:

Server selects and formats context information for prompt sentence generation.

Server chooses a subset of highly relevant candidate facilities based on the combined scores and extracts key attributes, such as name, specialty category, main therapies, and notable constraints (for example, evening hours). Server formats these attributes as short textual descriptions that can be embedded into a prompt sentence.

Input: Unified candidate list with scores and facility attributes.

Output: A compact textual representation of selected facilities and their attributes.

### Step 13:

Server generates a prompt sentence for the generative AI model.

Server composes a prompt sentence that includes (i) a summary of the user's symptoms and preferences derived from the abstract matters and (ii) the formatted descriptions of candidate facilities, followed by explicit instructions to the model. For example, the server may generate:

“A patient reports chronic low back pain that has persisted for more than 6 months and prefers exercise therapy instead of medication. The patient needs a facility near the workplace and is available on weekday evenings. The following facilities are candidates: Facility A specializes in spinal rehabilitation and offers evening exercise programs; Facility B provides general physical therapy with limited evening hours; Facility C focuses on manual therapy without structured exercise programs. Please select the most suitable facilities for this patient and explain in simple language why each selected facility is appropriate.”

Input: Abstract matters, textual facility descriptions, and instruction templates stored in memory.

Output: A complete prompt sentence string ready for submission to the generative AI model.

### Step 14:

Server submits the prompt sentence to the generative AI model and obtains generated information.

Server sends the prompt sentence via an application programming interface to a generative information processing apparatus that hosts a generative AI model, such as a transformer-based language model. The apparatus executes the model, performs attention-based computations over the tokenized prompt, and generates an output sequence. Server receives the output text, which includes selected facility names or descriptions and explanatory sentences.

Input: Prompt sentence string and model configuration parameters (such as maximum length and sampling strategy).

Output: Generated text containing selection results and explanatory information.

### Step 15:

Server parses the generated information and aligns it with candidate facilities.

Server processes the generated text using pattern matching and, optionally, secondary tokenization to identify references to facility names or attributes. Server associates each mentioned facility with its corresponding record in the candidate list. If the generative AI model outputs suggested rankings or qualitative descriptors (for example, “most suitable,” “good alternative”), server maps these descriptors to numerical weights.

Input: Generated text from the generative AI model and the unified candidate facility list.

Output: An annotated facility list where each facility has associated explanatory text and possibly an updated suitability score.

### Step 16:

Server computes final ranking, filtering, and integration of healthcare service point information.

Server recalculates a final score for each facility by combining the earlier combined score with weights derived from the generated information, such as emphasis on exercise therapy or suitability for evening availability. Server filters out facilities below a threshold score and sorts the remaining facilities according to the final score. Server then constructs integrated healthcare service point information that includes, for each facility, core attributes and AI-generated explanatory comments.

Input: Annotated facility list with original scores and AI-derived weights.

Output: Integrated healthcare service point information with a ranked and filtered facility set and attached explanations.

### Step 17:

Server optionally generates or updates an individualized treatment plan.

Server uses the abstract matters and integrated facility information to compose another prompt sentence that requests a time-series treatment schedule and lifestyle guidance. For example, the server may generate:

“For a patient with chronic low back pain who wants to avoid surgery and medications and has access to the selected exercise-focused facility, generate a detailed three-month treatment schedule including weekly supervised exercise sessions and daily home exercises, and provide lifestyle guidance to support pain reduction.”

Server submits this prompt sentence to the generative AI model, receives a structured textual treatment plan, and attaches it to the integrated healthcare service point information.

Input: Abstract matters, integrated facility information, and a treatment-plan instruction template.

Output: An individualized treatment plan represented as a time-ordered sequence of recommended actions and guidance.

### Step 18:

Server transmits integrated information and optional treatment plan to the terminal.

Server packages the ranked facility list, explanatory texts, and treatment plan into a response message formatted for client consumption. Server sends this message to the terminal over the network.

Input: Integrated healthcare service point information and individualized treatment plan data structures.

Output: A response payload containing displayable data delivered to the terminal.

### Step 19:

Terminal renders the results and allows user interaction.

Terminal parses the response payload, builds visual components such as a scrollable facility list and detail views, and displays facility names, treatment categories, and explanatory comments. Terminal may display the treatment plan as a calendar or list of sessions. User reviews the information and selects one or more facilities or indicates acceptance or rejection of the treatment plan, using buttons or touch gestures.

Input: Response payload from the server and user interaction events (scroll, tap, selection).

Output: Displayed information on the screen and new user commands or feedback actions.

### Step 20:

Terminal collects evaluation information and progress information and sends them to the server.

User provides feedback on the selected facility and the treatment plan, such as pain level ratings, adherence information, and comments on difficulties. Terminal records these as evaluation information and progress information and transmits them to the server.

Input: User-entered feedback, ratings, and textual comments.

Output: A feedback payload containing evaluation information and progress information delivered to the server.

### Step 21:

Server updates user profiles and generates refinement prompts for the generative AI model.

Server stores evaluation information and progress information in a user-specific profile.

Server then constructs a refinement prompt sentence that includes original treatment goals, current progress, and reported issues. For example, the server may generate:

“The patient with chronic low back pain has followed the recommended exercise therapy plan for 4 weeks and reports moderate pain relief but difficulty with certain exercises due to knee discomfort. Based on the original plan and the following progress notes, propose modifications to reduce knee strain while maintaining effectiveness for low back pain.” Server submits this refinement prompt to the generative AI model, obtains an improvement proposal, and updates the individualized treatment plan accordingly.

Input: Stored user profile, existing treatment plan, evaluation information, and progress information.

Output: An updated treatment plan and associated explanatory notes, which are then available for transmission to the terminal in a subsequent response.

### Application Example 1

Description follows regarding a flow of the specific processing in an Application Example 1. The units of the system described below are implemented by the data processing device 12 and the smart device 14. The data processing device 12 is called a “server” and the smart device 14 is called a “terminal”.

Conventional medical facility search and reservation systems typically rely on fixed rule sets, static keyword matching, and manually curated mappings between patient inputs and facility categories. Such systems are limited in their ability to understand free-form natural language descriptions of symptoms and treatment requests, and therefore often fail to surface facilities that are clinically appropriate for nuanced or complex conditions such as chronic or atypical low back pain. Moreover, in many existing implementations, the pipeline from user input to search result is fragmented: natural language processing, medical categorization, geographic filtering, ranking, and reservation handling are implemented as loosely coupled modules that do not adaptively share semantic information, which leads to inefficient processing, brittle behavior, and suboptimal use of computing resources.

In addition, known systems generally treat location-based search and clinical relevance as separate concerns. Geographic filtering is often performed as a simple distance-based query that is agnostic to the semantic content of the patient's description and to the relative priority or suitability of different facility types. As a result, computing resources are spent retrieving and ranking large candidate sets that are not well aligned with the user's actual medical needs, and the system may return facilities that are geographically close but medically inappropriate.

Further, while generative AI models have become available as general-purpose services, conventional systems do not tightly integrate such models into the core computation pipeline in a way that produces machine-readable, structured outputs that can directly drive downstream database queries, ranking logic, and reservation processing. Generative models are often used merely as auxiliary recommendation engines producing unstructured text that requires ad hoc parsing. This causes increased latency, inconsistent mappings from textual recommendations to database schemas, and higher error rates, thereby degrading the overall efficiency and reliability of the computer system.

There is therefore a need for a computer-implemented system that improves the way computing components process natural language symptom information and request information, generates structured prompt sentences for a generative AI model, and uses the model's structured output as a first-class computational signal to dynamically configure database search conditions, geographic constraints, and ranking logic. By tightly coupling prompt generation, generative AI inference, structured parsing, and geo-aware facility search within a unified processing pipeline, the system should reduce redundant computation, improve the accuracy and relevance of search results, and enable robust, low-latency reservation workflows. The technical problem can thus be viewed as improving the functioning of the computer system itself—specifically, improving natural language understanding, structured data derivation, and geo-personalized search and reservation processing for medical facilities—through a novel integration of generative AI models with database and location-based computation.

The specific processing by the specific processing unit 290 of the data processing device 12 in Application Example 1 is realized by the following means.

The present invention provides a server comprising a processor configured to present, on an information input/output device, an input screen through which a user inputs symptom information and request information, to acquire position information from the information input/output device and temporarily store the acquired position information in association with the symptom information and the request information as acquisition data, and to analyze natural language included in the acquisition data by using a natural language processing method so as to extract a term relating to a symptom and a term relating to a treatment and generate a prompt sentence including an extraction result as input to a generative AI model; the processor is further configured to receive structured output information from the generative AI model in response to the prompt sentence, to convert attribute information of a medical service provider included in the structured output information into internal classification information, to determine facility classification information based on the internal classification information, to search medical facility information stored in an information storage device on the basis of the facility classification information and the position information so as to extract a set of candidate medical facilities that satisfy a reliability condition and a distance condition, and to rank the set of candidate medical facilities on the basis of priority information derived from the structured output information, evaluation information, and distance information to generate search result information as structured data; the processor is further configured to associate the search result information with map information corresponding to the position information, to transmit display data to the information input/output device to cause the information input/output device to perform map display and list display of the candidate medical facilities, and to receive reservation information for a selected medical facility from the information input/output device, generate a reservation record based on the reservation information, and notify the selected medical facility of the reservation record. This enables the computer system to transform free-form natural language inputs and location data into machine-readable, semantically rich facility classification and ranking criteria, to perform geo-personalized search and reservation processing in a single integrated computational pipeline, to reduce redundant processing and ad hoc parsing, and to improve the accuracy, efficiency, and robustness of medical facility search and reservation operations executed by the server.

The term “information input/output device” refers to an electronic apparatus that provides a user interface for inputting and outputting information, including but not limited to a handheld terminal, a mobile computing device, a tablet device, a desktop terminal, or any other display-and-input capable device connected to the server.

The term “symptom information” refers to data representing a physical or mental condition of a user, including free-form natural language descriptions, selected items from predefined lists, or any structured or unstructured input indicating a medical complaint.

The term “request information” refers to data representing a preference or requirement of a user regarding treatment, consultation type, facility characteristics, schedule, or any other condition related to receiving medical or health-related services.

The term “position information” refers to location data indicating a geographic position of the information input/output device or user, including coordinates such as latitude and longitude, or any other data that can be used to determine a geographic location.

The term “acquisition data” refers to a data set obtained by the server that associates the symptom information and the request information with the position information, and that is temporarily or persistently stored for subsequent processing.

The term “natural language processing method” refers to a computational technique or algorithm that analyzes human language text or speech to identify linguistic structures, extract semantic information, or otherwise derive structured data from unstructured language.

The term “term relating to a symptom” refers to a linguistic element, such as a word or phrase, extracted from natural language input that denotes or describes a physical or mental condition, complaint, or medical problem.

The term “term relating to a treatment” refers to a linguistic element, such as a word or phrase, extracted from natural language input that denotes or describes a treatment method, therapy, medical specialty, or intervention desired or relevant to the user.

The term “prompt sentence” refers to a text string or set of text strings generated by the server that includes information derived from the symptom information and the request information, and that is formatted as an instruction or query to a generative AI model.

The term “generative AI model” refers to a machine-learned model configured to generate output data, including text or structured data, in response to input data such as a prompt sentence, where the model has been trained on data sets to produce contextually appropriate responses.

The term “structured output information” refers to data produced by the generative AI model in a machine-readable structured format, such as a data object, list, or record having predetermined keys, fields, or labels.

The term “attribute information of a medical service provider” refers to data elements that describe characteristics of a medical facility or practitioner, including but not limited to type of service, specialty, available treatments, equipment, certifications, or quality indicators.

The term “internal classification information” refers to data that represents a standardized or normalized category or code used internally by the server to classify medical service providers or facilities based on attribute information.

The term “facility classification information” refers to information derived from the internal classification information that identifies one or more categories or types of medical facilities to be targeted in a search process.

The term “information storage device” refers to a hardware or software component capable of storing data, including but not limited to a database system, a storage medium, or a memory device, used to store medical facility information and other records.

The term “medical facility information” refers to data that describes medical or health-related institutions, including facility identifiers, names, addresses, location coordinates, services offered, specialties, certifications, ratings, and other related metadata.

The term “reliability condition” refers to one or more criteria applied to medical facility information to ensure a level of trustworthiness or quality, such as minimum rating thresholds, certification requirements, review counts, or verification status.

The term “distance condition” refers to one or more criteria applied to medical facility information based on spatial proximity, such as a maximum allowable distance from the position information or a defined search radius.

The term “candidate medical facilities” refers to one or more medical facilities selected from medical facility information that satisfy at least the facility classification information and the reliability condition and the distance condition.

The term “priority information” refers to data that indicates a relative importance or ranking level of a medical facility or facility category, including priority levels or scores derived from the structured output information of the generative AI model.

The term “evaluation information” refers to data used to assess the quality or suitability of a medical facility, such as ratings, reviews, historical performance indicators, or other measures of user satisfaction or clinical relevance.

The term “distance information” refers to data representing a spatial measure between the position information and the location of a medical facility, such as a calculated distance or travel time.

The term “search result information” refers to structured data generated by the server that represents the outcome of a search process for candidate medical facilities, including identifiers, attributes, rankings, and associated geographic data.

The term “map information” refers to geographic representation data, including digital map tiles, vectors, or coordinate-based imagery, that enables graphical display of locations corresponding to position information and facility locations.

The term “display data” refers to formatted information transmitted from the server to the information input/output device for presentation to a user, including map display elements, list entries, and associated graphical or textual content.

The term “reservation information” refers to data representing a user's request to schedule an appointment with a selected medical facility, including at least a facility identifier, a date, a time, and optionally additional notes or user identifiers.

The term “reservation record” refers to a structured data entry created by the server that stores details of a confirmed or pending reservation with a medical facility, including reservation identifiers, facility identifiers, user identifiers, time slots, and status information.

In one embodiment, a server executes a medical facility search and reservation system on general-purpose computing hardware. The server uses a processor, a main memory, a non-volatile storage device, and a network interface. The server runs an operating system such as a Unix-like operating system and application software implemented, for example, in a programming language such as Python. The server uses a web application framework such as a generic web framework, a relational database management system such as a generic relational database, and a geographic extension module or library. The server communicates with one or more terminals over a network using a protocol such as HTTPS. A terminal comprises a portable computing device such as a smartphone or a tablet including a central processing unit, a display, an input unit, a wireless communication interface, and a positioning module such as a global navigation satellite system receiver. The terminal runs an operating system such as a mobile operating system and a client application implemented as a native application or a web-based application. The terminal provides an input/output interface for a user to input symptom information and request information, and for displaying search results and reservation information.

The server provides, to the terminal, user interface data that describes input screens containing fields for symptom information and request information. The terminal renders these screens on a display by using user interface libraries provided by the operating system. The user inputs symptom information as free-form text describing, for example, low back pain, and selects request information such as preferred treatment types or facility characteristics. The terminal stores the symptom information and the request information in an internal data structure, such as key-value pairs or an object, and associates them with locally acquired position information.

The terminal acquires position information by invoking location APIs provided by the operating system that access the positioning module. The terminal obtains, for example, latitude, longitude, and accuracy values, and stores them in a memory area. The terminal combines the symptom information, the request information, and the position information into a structured payload, such as a hierarchical data object, and transmits the payload to the server via the network interface using an encrypted communication channel.

The server receives the payload and stores the received data in the main memory. The server writes an entry into the relational database, in a table that stores query logs, including the symptom information, the request information, the position information, and metadata such as timestamps and user identifiers. The server then performs natural language processing on the symptom information and the request information. The server can use a text processing library implementing tokenization, part-of-speech tagging, and named-entity recognition. The server extracts terms relating to symptoms, such as “chronic low back pain” or “leg numbness,” and terms relating to treatments, such as “exercise therapy,” “manual therapy,” or “orthopedic consultation.” The server further normalizes these terms by mapping synonyms and variants to internal canonical representations using a medical terminology dictionary stored in the database.

The server generates a prompt sentence for a generative AI model by inserting the normalized symptom terms and treatment-related terms into a template. The server also appends explicit instructions for structured output. For example, the server generates a prompt sentence such as:

“Patient symptoms: chronic low back pain, worse when sitting and standing for long periods. Desired treatment: exercise therapy and manual therapy. Based on this information, please output a list of suitable facility categories. For each category, describe facility type, required specialty, recommended treatments, and priority level, in a format that can be easily parsed.” In another example, the server generates a prompt sentence such as:

“Patient symptoms: low back pain with intermittent numbness in the right leg; duration 6 months. Desired treatment: orthopedic consultation and diagnostic imaging such as MRI. Based on this information, please list appropriate facility types and their required specialties and mention whether advanced imaging equipment is important, and indicate a priority level for each facility type.”

The server provides the prompt sentence as an input to a generative AI model deployed as a service. In one embodiment, the generative AI model is implemented as a transformer-type neural network trained on a large corpus of medical and general text. The model architecture includes multiple self-attention layers, feed-forward layers, and layer normalization components. The model uses token embeddings, positional encodings, and multi-head attention mechanisms to compute context-aware representations of the input prompt sentence. During inference, the model receives the prompt sentence tokenized into subword units, processes the tokens through the attention layers, and outputs a sequence of tokens representing the result.

The server configures the generative AI model with parameters such as a low sampling temperature to favor deterministic and stable outputs, a maximum token limit to control response length, and a system-level instruction to bias outputs toward structured, machine-readable content. During training, such a model can be trained using supervised learning and reinforcement learning with human feedback, with loss functions such as cross-entropy loss for next-token prediction and additional regularization. Weights of the neural network are updated during training by gradient-based optimization methods. Although the server in operation time does not modify the trained weights, the server exploits the trained internal representations and learned attention patterns to generate semantically rich and medically informed output.

The server receives the output from the generative AI model as a text sequence and parses it into structured output information. The server expects that the generative AI model adheres to the constraints specified in the prompt sentence, such as listing facility types with attributes and priority levels in a predictable pattern. The server uses deterministic parsing rules, such as splitting by line breaks and specific markers, or by applying pattern matching expressions, to extract fields such as facility type, required specialty, recommended treatments, and priority level. The server thereby converts the generative output into a structured internal representation, such as a list of records.

The server maps facility types indicated in the structured output information to internal classification information stored in the database. The server maintains a mapping table that associates human-readable facility types (for example, “orthopedic clinic,” “rehabilitation center,” “sports medicine clinic,” or “pain clinic”) with standardized facility category codes. The server uses exact string matching, normalized matching, or approximate matching algorithms to align the generative AI model output with internal codes. The server thus determines facility classification information for use in downstream database queries.

The server queries the medical facility information stored in the relational database. The database maintains tables for facilities, categories, location coordinates, certifications, average ratings, user review counts, and available treatments. The server constructs a database query that filters facilities by facility category codes derived from the facility classification information, and by reliability conditions such as minimum rating thresholds or certification flags. The server also incorporates distance conditions. The server either uses spatial extensions in the database or an external geographic computation library to calculate distances between the user's position and each facility. The server restricts results to facilities within a predetermined radius or dynamic radius based on the priority information.

The server evaluates a priority score for each candidate facility using a multi-factor scoring function. The server incorporates priority information derived from the generative AI model output, evaluation information such as ratings and review counts, and distance information obtained from geographic calculation. The server can apply a weighted sum or another ranking algorithm, with weights configured to emphasize medical suitability for the condition and then geographic proximity. In some embodiments, the server can adjust these weights dynamically based on the type of symptom or request, thereby reducing computational cost by focusing ranking on fewer, highly relevant candidates.

The server generates search result information as structured data. The search result information includes, for each candidate facility, a facility identifier, name, address, geographic coordinates, category name, attributes such as available therapies, and the computed ranking score. The server also includes additional notes derived from the structured output information of the generative AI model, such as whether the facility is suited for chronic low back pain requiring exercise therapy or for patients needing diagnostic imaging. The server stores the search result information in memory and optionally logs a summary in the database for auditing and performance tuning.

The server associates the search result information with map information. The server retrieves base map tiles or vector map data from a map service or from locally stored map data. The server computes marker positions on the map corresponding to each facility's coordinates and may cluster markers if facilities are densely located. The server constructs display data that includes marker definitions, facility metadata for list display, and any route or distance information to be displayed.

The server transmits the display data to the terminal via the network interface. The terminal receives the display data, parses the structured data, and updates its user interface. The terminal uses its graphics subsystem and map rendering libraries to draw map elements and facility markers on the display. The terminal also generates a scrollable list view of facilities ordered by the ranking provided by the server. By offloading the computationally intensive ranking and classification to the server and sending only structured display data, this architecture reduces communication payload size and avoids redundant recalculation at the terminal, thereby lowering communication burden and improving response time.

The user interacts with the displayed facilities by tapping on a list item or a map marker. The terminal identifies the selected facility based on identifiers in the display data and presents detail information. If the user chooses to make a reservation, the terminal collects reservation information such as date, time slot, and additional notes. The terminal forms a reservation payload and sends it to the server.

The server receives reservation information and validates it against business rules and schedule data. The server accesses a reservations table in the database and uses transactional operations to avoid conflicts. The server checks that the requested time slot is not already booked and that the facility is available at that time. Upon confirmation, the server generates a reservation record including reservation ID, facility ID, user ID, date, time, and status. The server writes the reservation record into the database and sends confirmation data back to the terminal. The server may also notify an external facility-side system via an application programming interface.

By structuring the system as described, the server improves computer technology in several ways. The server reduces processing redundancy by using the generative AI model as a semantic front end that outputs structured classifications compatible with the database schema, thereby avoiding complex, hand-written rule sets and expensive iterative queries. The server improves accuracy of facility matching because the generative AI model captures context and implicit medical relationships between symptoms and facility types beyond simple keyword matching. The server improves computational efficiency by dynamically narrowing the search space based on facility classification information and priority information before performing geographic distance calculations and ranking, which reduces the number of distance computations and database operations.

The server additionally improves data management by enforcing consistent internal classification information and by logging mappings between natural language inputs, generative model outputs, and final facility selections. This allows optimization of indexing strategies in the database and tuning of the scoring function, which results in lower query latency and more predictable resource utilization. The integrated pipeline from prompt sentence generation, generative AI inference, structured parsing, classification, and ranking is specifically arranged to operate on particular data structures and to generate outputs that directly drive map-based visualization and reservation processing in real time.

The generative AI model in this system uses an internal attention mechanism that identifies relationships between symptom descriptors and treatment-related phrases, which are then reflected in the structured output information. Unlike a human expert manually reading text and suggesting facility types, the model performs a high-dimensional vector computation that scales to large numbers of users simultaneously and provides consistent mapping to the internal classification schema. This non-conventional use of the model, combined with explicit prompt constraints and deterministic parsing rules, results in a computer system that performs natural language understanding and facility classification with lower error rates and improved throughput compared to rule-based systems.

In alternative embodiments, the server can deploy different types of generative AI models, such as encoder-decoder architectures or models fine-tuned specifically on healthcare-related data. The server can also vary the structure of the prompt sentence to include additional context, such as severity scores, prior treatments, or contraindications. For example, the server can generate another prompt sentence such as:

“Patient symptoms: acute low back pain after lifting heavy objects; pain level 8 out of 10; difficulty bending. Desired outcome: rapid pain relief and safe return to work. Based on this information, list suitable facility types and indicate which should be prioritized, considering facilities that provide urgent assessment and pain management.”

The server may further implement caching mechanisms that store generative AI model outputs for common symptom patterns, reducing inference calls and thus lowering latency and computational load. The server can also adjust the prompt sentence based on historical feedback stored in the database, thereby incrementally improving prompt design in a way that optimizes structured output consistency.

In another embodiment, the terminal can be implemented as a stationary kiosk located in a medical facility or public area. The terminal can acquire position information as a fixed location and still use the described pipeline to classify symptoms and suggest departments within the same or nearby facilities. In yet another embodiment, multiple servers can operate in a distributed architecture, where one server specializes in prompt generation and generative AI inference, and another server specializes in geographic search and ranking. The servers can exchange structured intermediate data over a secure internal network, enabling scalability and fault tolerance.

Because the server uses specifically structured prompt sentences, model configuration parameters, deterministic parsing routines, and facility classification mappings that are tightly integrated with the database and map rendering pipeline, the system goes beyond generic automation of human tasks. The system introduces a particular technical arrangement of computational components that improves the functioning of the computer system itself in terms of processing speed, accuracy, data consistency, and resource utilization.

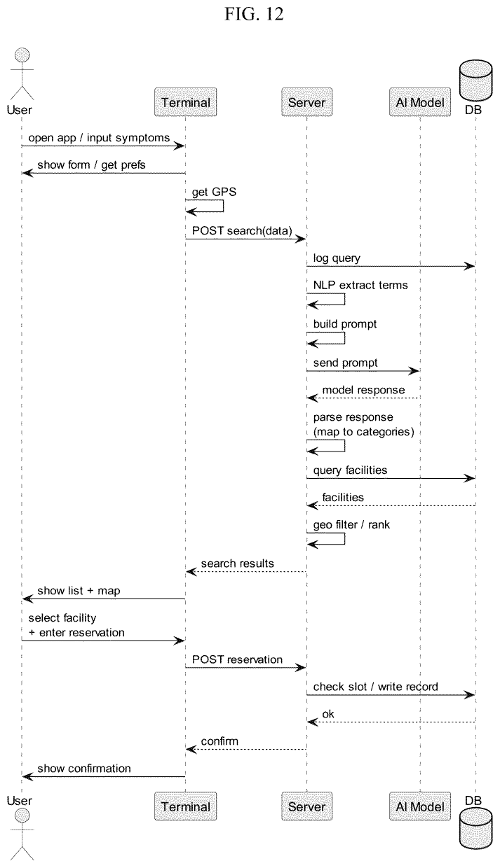

The following describes the processing flow using FIG. 12.

### Step 1:

User launches the application on the terminal and views an input screen. User inputs symptom information as natural language text (input: free-form text such as “chronic low back pain, worse when sitting”) and selects request information such as preferred treatment (“exercise therapy,” “manual therapy”) using selection widgets. Terminal receives these inputs and stores them in memory as structured fields (output: a local data object containing symptom_text and request_options).

### Step 2:

Terminal acquires position information by invoking an operating system location API. Terminal receives raw GPS or network-based coordinates (input: sensor readings from a positioning module) and converts them into numeric latitude and longitude values with an accuracy indicator (output: location_data containing latitude, longitude, and accuracy). Terminal then combines symptom_text, request_options, and location_data into a structured payload (output: search_payload object).

### Step 3:

Terminal transmits the search_payload to the server over a network connection using an HTTPS POST request. The input to this step is the search_payload object held in the terminal's memory. Terminal serializes the payload into a transport format such as JSON and sends it via the communication interface (output: HTTP request message containing serialized search_payload).

### Step 4:

Server receives the HTTP request at a predefined endpoint. The input to this step is the HTTP request message from the terminal. Server validates the request headers, parses the message body to reconstruct the search_payload, and performs basic schema validation (for example, checking for presence of symptom_text, request_options, and location_data). Server then writes a record into a query log table in a database (output: normalized_query_record stored in persistent storage and an in-memory representation of the same record).

### Step 5:

Server executes a natural language processing routine on the symptom_text and request_options. The input to this step is the text fields from the normalized_query_record. Server tokenizes the text, applies part-of-speech tagging, and performs named-entity recognition to detect symptom-related and treatment-related terms. Server then maps recognized terms to canonical forms using a medical terminology table (for example, mapping “backache” to “low back pain”) (output: extracted_symptom_terms and extracted_treatment_terms as lists of normalized tokens).

### Step 6:

Server generates a prompt sentence for a generative AI model. The input to this step is extracted_symptom_terms and extracted_treatment_terms. Server constructs a natural language instruction by inserting these terms into a template and appending constraints on the desired output format. Server performs string concatenation and formatting operations to produce a continuous text sequence. For example, server generates: “Patient symptoms: chronic low back pain, worse when sitting and standing for long periods. Desired treatment: exercise therapy and manual therapy. Based on this information, please output a list of suitable facility categories. For each category, describe facility type, required specialty, recommended treatments, and priority level, in a format that can be easily parsed.” (output: prompt_sentence string).

### Step 7:

Server sends the prompt_sentence to a generative AI model endpoint. The input to this step is the prompt_sentence. Server wraps the prompt_sentence into a request structure including model parameters such as temperature, maximum token count, and role descriptors. Server serializes this structure and transmits it over a secure connection to the generative AI model service. Server then waits for a response (output: inference_request message and, after remote computation, a model_response text sequence).

### Step 8:

Server processes the model_response returned by the generative AI model. The input to this step is the model_response text. Server first checks that the text conforms to the requested structure (for example, one category per line or tagged sections). Server then applies pattern matching and parsing routines to segment the response into records, extract facility_type, required_specialty, recommended_treatments, and priority_level fields, and normalize the strings (output: structured_model_output as a list or array of facility-type records suitable for programmatic use).

### Step 9:

Server maps the facility_type values in structured_model_output to internal classification codes used in the database. The input to this step is structured_model_output and a facility mapping table stored in the database. Server executes lookup operations that compare facility_type strings to entries in the mapping table; if an approximate match is needed, server may use string similarity calculations. Server assigns an internal category code to each facility_type (output: facility_classification_info consisting of internal category codes and associated priority information).

### Step 10:

Server queries the medical facility database using facility_classification_info and reliability conditions. The input to this step is facility_classification_info and database tables containing facility records, ratings, certifications, and treatment capabilities. Server constructs a query that selects facilities whose category_code is in the set of internal category codes and whose reliability metrics (for example, rating score, certification flag) meet predefined thresholds. Server executes the query and retrieves a candidate set of facilities (output: candidate facilities list, each item containing facility attributes and location coordinates).

### Step 11:

Server refines the candidate_facilities list using the position information from the user. The input to this step is candidate_facilities and location_data from the original search_payload. Server calculates the distance between the user's coordinates and each facility's coordinates using a geographic distance function. Server discards facilities outside a configured or dynamically determined radius and attaches distance km values to the remaining candidates (output: geo_filtered_facilities list, each element containing facility attributes and computed distance_km).

### Step 12:

Server computes a ranking score for each facility in geo_filtered_facilities. The input to this step is geo_filtered_facilities, priority information from structured_model_output, and evaluation information from the database (for example, rating and review_count). Server applies a ranking algorithm, such as a weighted sum of normalized priority_level, rating, and inverse distance. Server then sorts the facilities in descending order of the ranking score to create an ordered list (output: ranked_facilities list with associated scores).

### Step 13:

Server generates search result information for display. The input to this step is ranked_facilities. Server extracts and formats fields such as facility identifier, name, address, category, treatments, rating, distance, and model-derived notes. Server packages this data into a structured response object optimized for rendering, and may include summary metadata such as total number of results and search radius (output: search_result_payload structured object).

### Step 14:

Server associates the search_result_payload with map information and transmits display data to the terminal. The input to this step is search_result payload and map coordinate data. Server computes map marker definitions for each facility and includes them in the response. Server serializes the search_result_payload into a transport format and returns it as an HTTP response to the terminal (output: HTTP response message containing display_data for map and list views).

### Step 15:

Terminal receives the HTTP response and updates the user interface. The input to this step is the response message containing display_data. Terminal parses the structured data into native objects and passes facility coordinate data to a map-rendering component. Terminal draws markers on the map, generates a list view sorted by ranking, and displays distances and ratings next to each facility (output: updated screen showing ranked facilities on a map and in a list).

### Step 16:

User reviews the displayed facilities and selects a desired medical facility. The input to this step is the visual display and associated user controls. User taps a list item or map marker corresponding to a facility of interest. Terminal captures the selection event, identifies the facility by its identifier in the display_data, and presents a detail screen (output: facility_selection data within the terminal's memory and a transition to a detailed facility view).

### Step 17:

User inputs reservation information for the selected facility. The input to this step is the facility_selection and the reservation interface fields. User chooses a date and time slot and may enter additional notes about symptoms. Terminal collects this information and validates that required fields are filled. Terminal then constructs a reservation_payload containing facility_id, desired date and time, user identifier, and notes (output: validated reservation_payload object).

### Step 18:

Terminal transmits the reservation_payload to the server. The input to this step is the reservation_payload object. Terminal serializes the payload and sends it using an HTTPS POST request to a reservation endpoint on the server. Terminal waits for a server response indicating success or failure (output: HTTP request message carrying reservation_payload).

### Step 19:

Server receives and processes the reservation request. The input to this step is the reservation payload from the terminal. Server validates that the facility exists, checks that the requested time is in the future, and performs a database query to detect any existing reservations at the same facility and time. Server uses transactional control to ensure consistency; if no conflict exists, server creates a new reservation record in the database with status set to confirmed (output: reservation_record stored in persistent storage and an in-memory confirmation result).

### Step 20:

Server sends a reservation confirmation to the terminal. The input to this step is the confirmation result and reservation_record. Server generates a confirmation object containing reservation ID, date, time, facility information, and any instructions, and serializes it into a response message. Server returns this message as an HTTP response (output: HTTP response message containing reservation_confirmation data).

### Step 21:

Terminal receives the reservation confirmation and updates the display and local storage. The input to this step is the reservation_confirmation data. Terminal parses the data, shows a confirmation screen with reservation details to the user, and stores the reservation in local storage for offline viewing. Terminal may also schedule a local reminder through the operating system (output: updated user interface with confirmation and a locally persisted reservation entry).

### Step 22:

User may later modify symptom information or request information and initiate a new search. The input to this step is the existing application state and updated user input. User edits the symptom description (for example, changing to “low back pain with intermittent numbness in the right leg”) and updates the preferred treatment (“orthopedic consultation and diagnostic imaging”). Terminal treats this new input as fresh symptom_text and request_options and repeats the sequence from Step 2 onward. The output of this step is a new search_payload that reflects the updated medical condition and preferences, leading to a new cycle of data processing and facility recommendation based on a newly generated prompt sentence and generative AI model output.

It is also possible to incorporate an emotion engine for estimating the user's emotions. That is, the specific processing unit 290 may estimate the user's emotions using an emotion identification model 59, and perform specific processing based on the estimated emotions.

### Example 2

Description follows regarding a flow of the specific processing in an Example 2. The units of the system described below are implemented by the data processing device 12 and the smart device 14. The data processing device 12 is called a “server” and the smart device 14 is called a “terminal”.

Conventional computer-implemented systems for generating treatment plans using machine learning or generative AI techniques suffer from several technical limitations at the level of data processing, prompt generation, and interaction between a client terminal and a remote AI computation resource.

First, conventional systems often pass raw or loosely structured patient data directly to a generative AI model without systematic preprocessing, normalization, or validation on the server side. As a result, the prompt sentences sent to the AI model may contain inconsistent encodings, missing mandatory fields, or ambiguous symptom descriptions, which leads to unstable inference behavior, unnecessary token consumption, and degraded generation quality. This lack of structured preprocessing increases computational load and network usage, and complicates reproducibility and traceability of AI outputs.

Second, many existing systems do not treat prompt construction as a first-class programmable artifact on the server, but rather rely on ad hoc string concatenation in the client or within the AI provider's interface. Such approaches make it difficult to manage prompt templates, inject structured constraint information (for example, safety constraints, modality restrictions, or formatting requirements), and dynamically tailor prompts per patient session. This results in an inefficient use of the generative AI model, inconsistent formatting of responses, and increased post-hoc manual adjustment by downstream applications or human operators.

Third, conventional architectures typically do not provide a robust server-side mechanism to manage iterative refinement cycles in which updated patient conditions, constraints, or feedback are incorporated into subsequent prompts in a traceable and machine-usable manner. Without associating each AI-generated treatment plan with a stable server-generated identifier and structured history, systems cannot reliably reconstruct the evolution of treatment plans, cannot programmatically adapt prompts based on prior failures or user feedback, and cannot achieve continuous improvement of generated content without excessive manual intervention.

Fourth, response formatting is frequently handled in a simplistic or purely client-side manner, leading to heterogeneous representations of AI outputs at different terminals. In the absence of consistent server-side formatting-such as programmatic division of AI output into defined logical sections and normalization of whitespace and control characters—downstream rendering components must implement redundant parsing logic. This increases client-side processing complexity, makes interoperability with other systems more difficult, and can result in display errors or misalignment of content across different devices. Accordingly, there is a need for an improved computer-implemented server architecture and processing pipeline that: (i) receives patient symptom and treatment request information from a terminal and converts it into validated structured data; (ii) systematically generates patient-specific prompt sentences from predefined templates while embedding constraint conditions and output formatting instructions; (iii) coordinates secure, parameterized interactions with a generative AI model; (iv) performs consistent server-side post-processing and logical segmentation of AI-generated treatment plans; and (v) manages iterative refinement based on updated conditions and feedback, all in a way that improves the efficiency, reliability, reproducibility, and interoperability of computer-based treatment plan generation.

The specific processing by the specific processing unit 290 of the data processing device 12 in Example 2 is realized by the following means.

The present invention provides a server comprising a processor configured to provide, to a terminal, an input screen on an information processing apparatus for allowing a user to input symptom information and treatment request information of a patient, to receive, from the terminal via a secure communication scheme, the symptom information and the treatment request information, to store the symptom information and the treatment request information as structured data, to generate identification information corresponding to the patient based on the structured data, to perform preprocessing on the structured data including character string normalization processing and mandatory item verification processing, to select a prompt sentence template based on a result of the preprocessing, the prompt sentence template being configured to receive the symptom information and the treatment request information, to generate a patient-specific prompt sentence by inserting the structured data into predetermined positions in the prompt sentence template, to add constraint condition information relating to treatment content and instruction information relating to an output format of a generation result to the patient-specific prompt sentence so as to construct an input prompt sentence for a generative AI model, to generate request data including the input prompt sentence and generation parameters, to transmit, via a network, a processing request to an external generative AI model using the request data, to acquire response data including treatment plan information generated by the generative AI model in response to the input prompt sentence, to extract the treatment plan information from the response data, to perform formatting processing on the treatment plan information including removal of leading and trailing whitespace, conversion of line break codes, and division of the treatment plan information into logical sections including at least a summary section, an exercise therapy section, and a lifestyle guidance section, to store formatted treatment plan information in association with the identification information, and to generate display data including the formatted treatment plan information and transmit the display data to the terminal so as to cause a display device of the terminal to display, for each patient, a customized treatment plan based on the formatted treatment plan information. This enables improvement of computer-based treatment plan generation by providing a unified server-side pipeline that normalizes and validates patient data, constructs structured and constraint-aware prompt sentences, controls interactions with a generative AI model through parameterized requests, and produces consistently formatted, logically segmented treatment plans that can be efficiently rendered on heterogeneous terminals and iteratively refined based on additional conditions and feedback.

The term “processor” refers to a hardware or virtual computing unit, such as a central processing unit or an execution core in a computing environment, that executes program instructions to perform the functions described in the present disclosure.

The term “terminal” refers to an information processing apparatus, such as a client device, that includes an input device and a display device and is configured to transmit data to, and receive data from, a server over a communication network.

The term “information processing apparatus” refers to an electronic device capable of executing software for data input, data display, and communication, including but not limited to a personal computer, a tablet device, or a mobile communication device.

The term “input screen” refers to a graphical user interface presented on a display device of a terminal that allows a user to input data by means of one or more input controls such as text fields, selection boxes, or buttons.

The term “symptom information” refers to text data or structured data indicating a physical or mental condition of a patient, including descriptions of pain, discomfort, duration of symptoms, location of symptoms, or other clinical manifestations.

The term “treatment request information” refers to data indicating a patient's or a medical worker's preferences or requests regarding a treatment modality, such as a desired therapy type, treatment intensity, or constraints on treatment methods.

The term “secure communication scheme” refers to a communication protocol that provides confidentiality, integrity, and authentication for data transmitted between a terminal and a server, such as a transport layer security protocol.

The term “structured data” refers to data formatted according to a predefined schema, such as a key-value structure or a record structure, enabling systematic storage, retrieval, and processing by a computing system.

The term “identification information” refers to data that uniquely associates stored information with a particular patient or session, such as a generated identifier string or code, without necessarily revealing personally identifiable information.

The term “preprocessing” refers to a set of data transformation operations performed on received data prior to further analysis or transmission, including validation, normalization, and formatting operations.

The term “character string normalization processing” refers to operations that standardize text data into a consistent representation, including but not limited to trimming whitespace, unifying character encodings, and standardizing character variants.

The term “mandatory item verification processing” refers to a validation procedure that checks whether required data fields are present and conform to specified constraints, such as type, length, or format requirements.

The term “prompt sentence template” refers to a text pattern including one or more placeholders for variable data, designed to be completed with structured data to form a full prompt sentence to be supplied to a generative AI model.

The term “patient-specific prompt sentence” refers to a prompt sentence obtained by inserting structured data corresponding to a particular patient into predetermined positions of a prompt sentence template.

The term “constraint condition information” refers to data indicating limitations or rules that should be reflected in content generated by a generative AI model, such as disallowed treatments, required safety considerations, or domain-specific restrictions.

The term “instruction information relating to an output format” refers to data specifying how a generative AI model should structure or present its generated output, including requests for headings, bullet lists, sections, or length constraints.

The term “input prompt sentence” refers to a complete textual instruction, including content conditions and formatting instructions, that is supplied to a generative AI model as input for generating a response.

The term “generative AI model” refers to a machine learning model that receives an input prompt and generates new text data in response, based on learned statistical patterns in training data, such as a language model or sequence-to-sequence model.

The term “request data” refers to a data structure transmitted from a server to a generative AI model, including at least an input prompt sentence and one or more generation parameters controlling the behavior of the model.

The term “generation parameters” refers to control values provided to a generative AI model to influence its output characteristics, such as temperature, maximum output length, or sampling strategy parameters.

The term “response data” refers to a data structure received from a generative AI model in reply to request data, including at least text data representing a generated treatment plan or other requested information.

The term “treatment plan information” refers to text data describing one or more proposed therapeutic actions or recommendations for a patient, including but not limited to exercise therapy content, lifestyle guidance, and related advice.

The term “formatting processing” refers to operations that modify the representation of text data without altering its substantive meaning, including whitespace normalization, control character normalization, and structural segmentation.

The term “line break code conversion” refers to a process of converting line ending characters in text data to a uniform format suitable for storage or display in a target environment.

The term “logical sections” refers to semantically distinct parts of a document or text, separated according to subject matter or function, such as a summary portion, a therapy portion, or a guidance portion.

The term “summary section” refers to a logical section of treatment plan information that provides a high-level overview of the treatment objectives or main recommendations. The term “exercise therapy section” refers to a logical section of treatment plan information that describes specific physical exercises or movement-based interventions recommended for the patient.

The term “lifestyle guidance section” refers to a logical section of treatment plan information that describes non-exercise behavioral recommendations, such as posture advice, daily habits, or environmental adjustments.

The term “formatted treatment plan information” refers to treatment plan information that has undergone formatting processing, including normalization and division into logical sections, to conform to a predetermined structure.

The term “display data” refers to data prepared for presentation on a display device, including formatted treatment plan information and, optionally, metadata for layout or rendering on a terminal.

The term “update information” refers to additional or revised data received after initial input, including new conditions, constraints, or corrections relating to a patient or a treatment plan. The term “additional condition information” refers to data indicating newly provided or previously unreported patient status, limitations, or context that may affect a recommended treatment plan.

The term “revised version of the treatment plan information” refers to treatment plan information generated by a generative AI model in response to an updated input prompt sentence that reflects update information.

The term “feedback information” refers to data indicating an evaluation or response to a treatment plan, provided by a patient or a medical worker, including indications of suitability, effectiveness, or problems encountered.

The term “history information” refers to data representing a sequence of past interactions, including prior treatment plan information, update information, and feedback information, associated with a patient or session.

The term “treatment success degree” refers to an indicator, explicit or implicit, of how well a treatment plan meets therapeutic objectives for a patient, including but not limited to symptom improvement, adherence, or satisfaction metrics.

In one embodiment, a server, a terminal, and a communication network cooperate to implement a system that generates and provides customized treatment plans using a generative AI model based on structured patient information. The server executes a program on a hardware platform such as a general-purpose server computer including at least one central processing unit (CPU), a main memory, a non-volatile storage device, and a network interface. The terminal is realized by an information processing apparatus such as a personal computer, a tablet device, or a mobile communication device, including an input device (e.g., keyboard, touch panel) and a display device (e.g., liquid crystal display, organic electroluminescent display). The communication network is realized by a wired or wireless network infrastructure including, for example, a router, a modem, and an external packet-switched network.

Server uses an operating system such as a UNIX-like operating system and executes middleware such as a web server framework and an application server. For example, server uses a web application framework implemented in a programming language such as Python (e.g., Django or Flask) or JavaScript (e.g., Node.js with Express) and uses a database management system such as a relational database. Server loads and executes an application program that implements the functions described in the claims.

Terminal uses a web browser such as a general-purpose web browser or a dedicated application to communicate with server. Terminal displays an input screen generated by server. The input screen is formed by a markup document and a script, for example an HTML document with JavaScript, or native user interface components in a mobile application. The input screen includes text input fields, selection lists, and buttons that allow user to input symptom information and treatment request information of a patient.

User uses terminal to input texts describing symptoms, such as “chronic lower back pain” or “pain increases when sitting for more than 30 minutes,” and to specify desired treatment modalities, such as “exercise therapy” or “home-based low-impact therapy.” User optionally inputs additional constraint information such as “avoid deep knee bending,” “no equipment,” or “maximum 20 minutes per day.” Terminal converts these inputs into structured data, for example a key-value representation in memory, and transmits the structured data to server through a secure communication scheme such as HTTPS based on transport layer security. Server receives the structured data and performs a series of data processing operations. Server performs character string normalization processing, including unifying character encoding to a single encoding format such as UTF-8, normalizing line endings, trimming leading and trailing whitespace, converting variant characters to a normalized form, and optionally transforming full-width characters to half-width characters. Server performs mandatory item verification processing by checking that required keys such as symptom information, treatment request information, and basic patient context (for example, age range or activity level if required by configuration) are present and satisfy predetermined constraints, such as non-empty string constraints and allowed value ranges. If verification fails, server generates error information and causes terminal to display an error message, thereby enforcing data integrity before interaction with the generative AI model.

Server generates identification information for each patient or each treatment-plan generation session. Server uses, for example, a random or pseudo-random identifier generation algorithm (such as a universally unique identifier generation algorithm) to create an identifier string. Server stores the identifier string and associates it with the structured symptom information and treatment request information in a database record. By storing the data in this structured manner, server ensures that each subsequent interaction with the generative AI model, including updates or refinements, can be unambiguously associated with a specific patient session, improving traceability and reproducibility of AI-generated output.

Server manages a collection of prompt sentence templates stored in a non-volatile storage device, for example as text files or database entries. Each prompt sentence template defines a pattern including fixed text and placeholders for insertion of patient-specific structured data, constraint condition information, and formatting instructions. For instance, server may maintain templates such as:

“Patient has [SYMPTOM_DESCRIPTION] and prefers [TREATMENT_PREFERENCE]. Please propose a detailed treatment plan including exercise therapy and lifestyle guidance, tailored to this patient.”

“Patient is [AGE] years old and has [SYMPTOM_DESCRIPTION]. Patient requests [TREATMENT_PREFERENCE]. Please provide an evidence-based weekly exercise program and daily lifestyle recommendations.”

Server selects an appropriate prompt sentence template based on attributes of the structured data. For example, if structured data contains a key indicating that the patient is mainly suffering from chronic pain and requests exercise therapy, server selects a template specifically designed for chronic pain and exercise therapy. If structured data includes an indication of comorbid conditions, server selects an extended template that explicitly incorporates multi-condition constraints.

Server constructs a patient-specific prompt sentence by inserting values from the structured data into the placeholders in the selected template. Server uses a string substitution mechanism, such as formatted string operations in the application program, to generate a complete text. For example, server generates a prompt sentence such as:

“The patient has chronic lower back pain and wishes to receive exercise therapy. Please propose a detailed exercise therapy program tailored to this patient, including specific exercises, frequency, duration, and precautions. Also provide daily lifestyle improvement advice to reduce lower back pain.”

In another example, server generates:

“The patient is a 45-year-old office worker with chronic lower back pain who sits for about 8 hours a day. The patient prefers home-based exercise therapy. Please propose a weekly exercise program and workplace stretching routine, and suggest ergonomic improvements for the desk environment.”

In still another example, server generates:

“The patient has chronic lower back pain and mild hypertension. Please propose a low-impact exercise therapy program that avoids excessive cardiovascular strain and includes warm-up, main exercises, and cool-down, as well as lifestyle guidance for both back pain and blood pressure control.”

Server further augments the patient-specific prompt sentence with constraint condition information and output format instructions. Server appends or inserts explicit instructions such as “do not suggest surgical procedures,” “avoid recommending exercises that involve deep knee bending,” “limit the session duration to 20 minutes per day,” or “output in sections with headings ‘Summary,’ ‘Exercise Therapy,’ and ‘Lifestyle Guidance’ and use bullet points under each section.” By embedding such constraints and formatting specifications directly into the prompt sentence, server shapes the behavior of the generative AI model in a deterministic, programmable manner that is not dependent on ad hoc human prompt crafting. Server prepares request data for a generative AI model. Server uses an application programming interface provided by a generative AI service. Server sets generation parameters, such as maximum number of output tokens, a temperature parameter controlling sampling randomness, a top-k or top-p parameter controlling candidate truncation, and optional system-level instructions if the model supports a role-based input interface. Server encapsulates the input prompt sentence and generation parameters in a request data structure in memory.

Server transmits the request data to an external generative AI model endpoint via the network interface. On the provider side, the generative AI model is implemented as a neural network, for example a transformer-based language model. The model includes an embedding layer that converts input tokens into high-dimensional vectors, a plurality of self-attention layers that compute context-aware representations, and a decoding mechanism that sequentially generates output tokens. The model parameters (weights) are obtained by offline training on large text corpora through supervised or self-supervised learning. During training, the model minimizes a loss function such as cross-entropy between predicted tokens and ground-truth tokens using an optimization algorithm such as stochastic gradient descent with adaptive moment estimation. Weights are updated iteratively based on backpropagation of gradients computed over mini-batches of training data. Data augmentation strategies such as random masking, sentence reordering, or synonym substitution may be employed to improve generalization. Although such training is performed outside the system at deployment time, server is configured to interact with the resulting trained model through the standardized request and response interface.

Server receives response data from the generative AI model. The response data includes text representing a generated treatment plan. Server extracts the text portion from the response data and performs formatting processing. Server removes leading and trailing whitespace, unifies line break codes to a standard representation, and splits the generated text into logical sections. For example, server uses explicit section headings if the model follows the requested output format (e.g., “Summary,” “Exercise Therapy,” “Lifestyle Guidance”) or uses additional parsing rules based on textual markers to identify and segment these sections. Server then stores the segmented and formatted treatment plan information in association with the previously generated identification information in the database.

This server-side segmentation and normalization provide several technical advantages. Server produces consistently formatted treatment-plan data even if terminals differ in their hardware or software capabilities. Because server imposes a uniform structure on AI outputs, terminals can remain relatively thin clients and can render complex treatment plans without implementing heavy parsing logic. This reduces client-side processing load, enables reuse of existing display components, and improves overall system interoperability. Moreover, server's normalization reduces redundant tokens and unnecessary formatting irregularities, reducing storage size and bandwidth requirements when treatment plans are retrieved or transmitted again.

Terminal receives display data that includes the formatted treatment plan information. Terminal renders the data on the display device using a graphical user interface. Terminal may use a web rendering engine, a graphical toolkit, or a native UI framework. Terminal presents sections such as “Summary,” “Exercise Therapy,” and “Lifestyle Guidance” as separate visual elements, and displays each exercise with its frequency, duration, and precautions. User reviews the treatment plan on terminal and may use print functions or export functions if implemented.

In another embodiment, server supports iterative refinement of treatment plans. User provides update information via terminal, including new conditions or constraints discovered after initial plan generation, such as “patient also experiences knee pain,” “avoid forward bending past 45 degrees,” or “patient can only exercise three times per week.” Terminal sends the update information to server, which records the update information in association with the structured data and the identifier. Server constructs a new patient-specific prompt sentence that incorporates both the original structured data and the new update information. For example, server generates a prompt sentence such as:

“Patient has chronic lower back pain and cannot perform deep knee bending due to knee pain. Please revise the previous exercise therapy program and propose alternatives that avoid knee strain while still addressing lower back pain.”

Server then constructs updated request data and again interacts with the generative AI model. By programmatically combining prior context and new constraints into a single input prompt sentence, server ensures that every refinement step is reproducible and auditable. This server-controlled prompt reconstruction is different from simply resubmitting new free-form text because server maintains explicit structured records of all relevant attributes and constraints, which reduces ambiguity and improves the determinism of AI responses.

In still another embodiment, server incorporates feedback information into the generation process. User, who may be a patient or a medical worker, provides evaluation data on the usefulness or appropriateness of a generated treatment plan, such as “stretch A increased pain,” “exercise B is too difficult,” or “patient strongly prefers standing exercises.” Terminal sends the feedback information to server. Server records the feedback information with the identification information and the corresponding treatment plan. When generating a new treatment plan, server forms a prompt sentence that explicitly references history information including past treatment plans and feedback. For example, server generates:

“Previously generated plan included exercises A, B, and C. The patient reported that exercise A increased pain and exercise B was too difficult, while exercise C was acceptable. Please generate a revised treatment plan that excludes A and B, introduces new low-impact alternatives, and retains exercises with similar intensity to C.”

Server sends this constructed prompt sentence to the generative AI model, which then generates a new treatment plan that is more aligned with the patient's responses. By embedding explicit feedback-derived constraints and references into the prompt sentence, server effectively encodes a non-trivial optimization process: iteratively steering the generative model toward higher suitability according to structured user feedback. This yields a technical effect of reducing the number of cycles needed to converge on an acceptable plan, thus reducing computation time and network traffic compared to naïve trial-and-error interactions.

The described architecture improves computer technology beyond mere automation of human tasks. Server implements a structured, template-based prompt generation mechanism, including normalization, validation, and semantic segmentation pipelines, which results in more stable and predictable interactions with the generative AI model. These pipeline steps reduce error rates caused by malformed or incomplete input, lower the amount of unnecessary data transferred, and improve computational efficiency by avoiding repeated generation of unstructured or unusable responses. Additionally, server's centralized storage of structured data and formatted outputs supports efficient indexing and retrieval, which enhances data management and reduces the overhead associated with complex query operations.

From a system perspective, the combination of structured prompt construction, parameterized API interaction, and deterministic post-processing constitutes a specific improvement to the way computer systems orchestrate large-scale language models. Instead of naïvely forwarding raw user text, server enforces a non-conventional sequence of technical operations: strict schema validation, controlled insertion into predefined templates, embedding of explicit constraint condition information and output instructions, and standardized segmentation of model outputs. These operations are specifically tailored to harness the underlying generative AI model's capabilities while mitigating its variability. By performing these steps at the server layer, the system achieves improved throughput and reliability in a multi-user environment, where many terminals may concurrently request treatment plans.

Alternative embodiments may vary the implementation of each module while preserving the underlying technical approach. In one variant, server employs different database technologies, such as a document-oriented database instead of a relational database, while still storing structured data and formatted treatment plans in association with identifiers. In another variant, server interacts with different kinds of generative AI models, such as encoder-decoder architectures or models fine-tuned on medical texts, provided that the model accepts a prompt sentence and returns generated text. In yet another variant, server includes an additional safety filter module that performs rule-based scanning of generated text to detect and remove prohibited content before storage and display, thereby further enhancing system reliability.

In all embodiments, server, terminal, and user interact such that user inputs patient-related information via terminal, server performs structured processing, prompt generation, interaction with a generative AI model, and standardized post-processing, and terminal presents the customized treatment plan to user. By defining specific data structures, processing steps, and model interaction methods, the system achieves concrete technical effects in terms of processing speed, output quality, error reduction, and efficient utilization of computational and communication resources, thereby enabling practical realization of AI-assisted treatment plan generation in a robust and scalable manner.

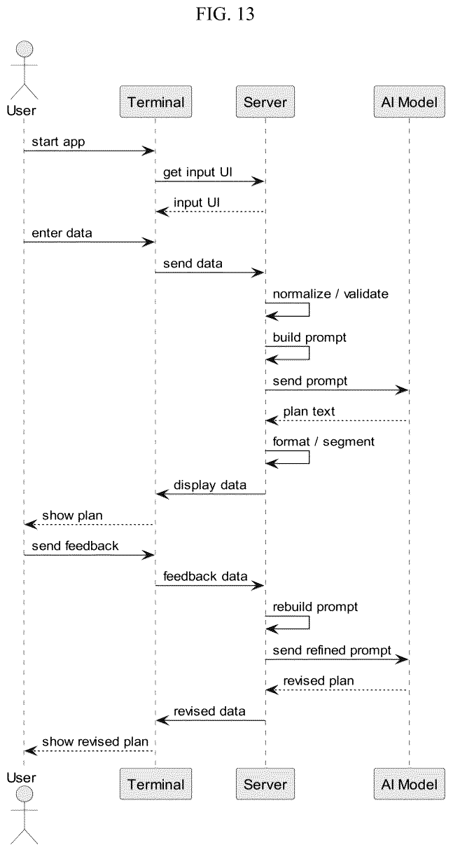

The following describes the processing flow using FIG. 13.

### Step 1:

User operates the terminal to start an application for treatment plan support.

User uses the input device of the terminal to launch a web browser or native application. The input of this step is a user action (application start command) and the output is an active application process on the terminal, ready to request an input screen from the server.

### Step 2:

Terminal requests an input screen from the server.

Terminal sends a request message to the server over a network connection, specifying that an input screen for patient symptom information and treatment request information is required. The input is the user's action to access the input screen, and the terminal converts this into a network request. The output is an HTTP or similar request packet addressed to the server.

### Step 3:

Server generates and returns the input screen to the terminal.

Server receives the request packet, parses the request, and selects an appropriate HTML/GUI template for the input screen. Server performs data processing by combining static templates with configuration data (e.g., field definitions) to form a complete markup document. The input to this step is the request from the terminal, and the output is a response containing the generated input screen content, which server sends back over the network.

### Step 4:

Terminal displays the input screen to the user.

Terminal receives the response from the server, decodes the markup and script data, and renders the input fields (for symptom information, treatment requests, and constraints) on the display device. The input is the screen definition data provided by the server, and the output is a visual user interface presented to user on the terminal's display.

### Step 5:

User inputs patient symptom information and treatment request information.

User enters text such as “chronic lower back pain” and selects options such as “exercise therapy” using the input controls. The input to this step is the visual interface and the user's physical interactions with the input device. The output is a set of raw input values (strings and selected options) held in the terminal's memory.

### Step 6:

Terminal validates and structures the input data.

Terminal applies validation logic to check that mandatory fields are filled and formats such as numbers or dates are correct. Terminal performs data processing by mapping each screen control to a key in a data structure and assembling a structured representation (e.g., a key-value map) of symptom information, treatment request information, and constraint information. The input is the raw user inputs, and the output is validated structured data ready for transmission.

### Step 7:

Terminal transmits the structured data to the server via secure communication.

Terminal uses a secure protocol (e.g., HTTPS) to serialize the structured data into a message format, encrypt the message, and send it through a communication device such as a router or modem. The input is the structured data in the terminal's memory, and the output is an encrypted request packet carrying the structured data to server.

### Step 8:

Server receives and decodes the structured data.

Server accepts the incoming network packets, decrypts them, parses the message format, and reconstructs the structured data object representing the patient's symptom information and treatment request information. The input is the encrypted request packet, and the output is a decoded and deserialized structured data object in server memory.

### Step 9:

Server performs preprocessing on the structured data.

Server applies character string normalization (e.g., trimming whitespace, standardizing line breaks, unifying encoding) and mandatory item verification (e.g., checking that symptom descriptions and treatment requests are present and non-empty). Server may also map synonyms or abbreviations to canonical terms using lookup tables. The input is the raw structured data received from terminal, and the output is normalized and verified structured data suitable for downstream processing.

### Step 10:

Server generates identification information and stores the structured data.

Server generates a unique identifier for the patient or session, using an identifier generation algorithm. Server stores the association between the identifier and the normalized structured data in a database. The input is the normalized structured data, and the outputs are the generated identifier and a database record linking the identifier to the structured data.

### Step 11:

Server selects an appropriate prompt sentence template.

Server analyzes attributes in the structured data, such as symptom type, requested therapy type, and additional constraints, and uses decision logic to select one prompt sentence template from a template repository. The input is the normalized structured data and the available templates; the output is a selected template definition with placeholders corresponding to data fields.

### Step 12:

Server generates a patient-specific prompt sentence.

Server performs string substitution by inserting values from the structured data (e.g., symptom description, age range, work style, preferred treatment) into the placeholders of the selected template. Server thereby constructs a complete patient-specific prompt sentence. The input is the selected template and the structured data; the output is a finished prompt sentence that describes the patient situation.

### Step 13:

Server augments the prompt sentence with constraint condition information and output format instructions.

Server appends or embeds additional instructions such as “avoid high-impact exercises,” “do not recommend surgery,” and “output in sections: Summary, Exercise Therapy, Lifestyle Guidance, using bullet points.” Server combines these constraints and formatting requirements with the patient-specific prompt sentence into a single input prompt sentence. The input is the initial prompt sentence and constraint settings; the output is an augmented input prompt sentence with explicit content and format specifications.

### Step 14:

Server constructs request data for the generative AI model.

Server packages the input prompt sentence together with generation parameters (e.g., maximum token count, temperature, sampling configuration) into a request object. Server performs data processing by encoding this object into a standardized request format, such as a JSON-like structure held in memory. The input is the input prompt sentence and parameter values; the output is a structured request data object prepared for transmission.

### Step 15:

Server transmits the request data to the generative AI model endpoint.

Server sends the request data over a network connection to an external endpoint that hosts the generative AI model. Server ensures that the data is serialized, encrypted, and addressed correctly. The input is the in-memory request data, and the output is an outgoing network request that initiates model inference.

### Step 16:

Server receives response data from the generative AI model.

Server obtains the model's response, decrypts the data if needed, and deserializes the response into an internal representation. The response includes text representing a generated treatment plan. The input is the raw response stream from the model endpoint; the output is a structured response object containing at least the generated text.

### Step 17:

Server extracts and normalizes the treatment plan information.

Server isolates the generated text portion from the response object and performs normalization operations such as removal of redundant whitespace and unification of line break codes. Server may also strip extraneous markers not needed for display. The input is the response object with the raw generated text; the output is a cleaned text string representing the treatment plan.

### Step 18:

Server segments the treatment plan into logical sections.

Server analyzes the normalized text and splits it into parts such as a summary section, an exercise therapy section, and a lifestyle guidance section. Server uses headings requested in the prompt, textual markers, or rules (e.g., detecting specific keywords) to determine section boundaries. The input is the normalized treatment plan text; the output is a structured representation of the treatment plan, for example a set of labeled sections each holding a subsection of the text.

### Step 19:

Server stores the formatted treatment plan associated with identification information.

Server writes the segmented and formatted treatment plan into a database and associates it with the identifier generated earlier. Server performs data operations to insert or update records so that each plan can be retrieved by identifier or other keys. The input is the structured treatment plan and the identifier; the output is a persistent database entry that represents the formatted treatment plan linked to the corresponding patient or session.

### Step 20:

Server generates display data for the terminal.

Server composes a display data object including the structured sections of the treatment plan and any necessary metadata for layout (e.g., section order, headings, bullet markers). Server performs serialization so that the display data can be transmitted over the network. The input is the database-resident or in-memory structured treatment plan; the output is a display-oriented data structure ready to be sent to terminal.

### Step 21:

Server transmits the display data to the terminal.

Server sends the display data to terminal using a secure communication channel. Server encapsulates the display data into a response message and writes it to the network interface. The input is the display data object; the output is a response packet reaching terminal that carries the structured treatment plan.

### Step 22:

Terminal receives and parses the display data.

Terminal obtains the response packet, decrypts and deserializes the content, and reconstructs the display data object in memory. The input is the packet from server; the output is an internal data structure representing sections of the treatment plan and display metadata.

### Step 23:

Terminal renders the treatment plan on the display device.

Terminal processes the display data by mapping each section (summary, exercise therapy, lifestyle guidance) to corresponding visual components on the screen, such as headings and bullet lists. Terminal executes layout calculations and draws text on the display. The input is the structured display data; the output is a rendered screen that shows a customized treatment plan to user.

### Step 24:

User reviews the treatment plan and optionally provides feedback or additional conditions.

User reads the displayed sections and may input additional comments or constraints such as “exercise B is too difficult” or “add stretches for the neck.” The input is the visual treatment plan and the user's evaluation; the output is new feedback data or updated condition data stored in the terminal's memory.

### Step 25:

Terminal structures and transmits update or feedback information to the server.

Terminal converts the new feedback or additional conditions into structured form, validates the content, and sends it to server using secure communication, similarly to prior data transmissions. The input is user-provided feedback text and choices; the output is a structured update message delivered to server for use in refinement of subsequent prompt sentences and treatment plans.

### Application Example 2

Description follows regarding a flow of the specific processing in an Application Example 2. The units of the system described below are implemented by the data processing device 12 and the smart device 14. The data processing device 12 is called a “server” and the smart device 14 is called a “terminal”.

Conventional computer-implemented healthcare support systems that generate treatment recommendations from free-text patient input suffer from multiple technical limitations. First, typical systems treat symptom text, sensor data, medical record data, and emotion-related input as separate, loosely coupled data streams. As a result, program logic executed by the processor cannot reliably form a unified, machine-usable representation of a patient state, which leads to unstable behavior of downstream models and inconsistent generation of treatment plans. Second, known systems generally invoke a generative AI model with static or manually authored prompts that are independent of runtime data such as real-time activity metrics, model-based plan effectiveness evaluations, or dynamically detected emotional states. This static prompting causes the processing pipeline to disregard newly available machine-generated indicators and thereby reduces the ability of the system to adapt plans in an efficient manner. Third, conventional systems often return the raw output text of a generative AI model directly to a client device, without converting the text into structured plan data that is linked in a traceable way to the underlying input signals and model configurations. This makes it difficult for the processor to perform subsequent computations such as plan adherence analysis, iterative re-planning, and auditable comparison among different plan versions.

A further technical problem arises in that many existing platforms use generic machine learning evaluation in a one-off batch mode. They do not feed the evaluation results back into the generative prompt construction mechanism at runtime. Consequently, the computational resources of the server are not leveraged to iteratively refine prompts and improve the quality and relevance of generated plans. This leads to redundant recomputation, duplicated communication with client devices, and inefficient use of model-inference capacity. Additionally, known systems lack a processor-level mechanism to integrate emotion estimation together with activity data into the core plan-generation loop. As a result, the presentation content of plans and the facility-selection criteria cannot be automatically tuned to a user's current emotional state, which limits the ability of the computer system to provide stable user engagement and predictable adherence, and often requires manual intervention by human operators.

Accordingly, there is a need for a computer-implemented system in which the processor (i) programmatically fuses natural language input, sensor information, medical record data, and emotion analysis results into structured user state information, (ii) automatically constructs and updates prompt sentences for a generative AI model on the basis of this user state information and machine-learned evaluation indicators, (iii) converts generated text into structured plan information suitable for further machine processing, and (iv) iteratively updates the plan information in a closed computational loop using execution status information, feedback information, and new emotion-related information. By addressing these technical problems in the way prompts are generated and consumed and in the way plan data are structured, the invention improves the operation of the computer system itself, including memory organization for patient state features, network usage for model calls, and processor-level control over generative AI inference cycles.

The specific processing by the specific processing unit 290 of the data processing device 12 in Application Example 2 is realized by the following means.

The present invention provides a server comprising a processor configured to acquire, via a user input/output device, natural language input information and sensor information including symptom information, request information, and emotion-related information of a user, append time information and location information to the acquired input information, and transmit and receive the information through a communication network; to analyze the acquired natural language input information by using a natural language processing method to perform morphological analysis and phrase extraction, extract phrases corresponding to the symptom information and the request information, analyze emotion from voice information or text information by using an emotion analysis method, and generate user state information including the symptom information, the request information, an emotion state, and activity information obtained from a biological information recording device; to search, on the basis of the user state information, data stored in a medical record information storage device and a facility information storage device, calculate candidate treatment facilities and effectiveness indicators of existing treatment plans, and generate structured data including the candidate treatment facilities and the effectiveness indicators; to generate, on the basis of the user state information and the structured data, a prompt sentence for causing a generative AI model to generate a response different for each user, input the prompt sentence into the generative AI model, and obtain generated text including treatment plan information or exercise plan information and explanatory information; to analyze the generated text to structure the generated text as plan information including implementation items on a daily basis or on a session basis, precaution information, motivation messages, and the candidate treatment facilities, store the structured plan information as history information for each user in a recording device, and transmit the structured plan information to the user input/output device; and to calculate achievement indicators and effectiveness indicators of the plan information on the basis of execution status information, feedback information, and additional emotion information acquired from the user input/output device, generate an update prompt sentence reflecting the indicators, and input the update prompt sentence into the generative AI model again to continuously update the plan information. This enables the computer system to transform heterogeneous patient-related data into a unified machine-usable state representation, to dynamically construct and refine prompt sentences for a generative AI model based on runtime analytical indicators, to convert generated free text into structured plan data suitable for further automated processing, and to execute an iterative, closed-loop adjustment of treatment and exercise plans without human intervention, thereby improving the efficiency, adaptability, and technical performance of the underlying information processing architecture.

The term “user input/output device” refers to a hardware and software interface apparatus, such as a terminal or client device, that enables a user to input information including natural language text, voice, images, and sensor data, and to receive information including plan information, messages, and recommendations from the server through a communication network.

The term “natural language input information” refers to information expressed in a human language, including but not limited to sentences, phrases, and words input by the user via text, voice, or other modalities, before being converted into a structured representation by a natural language processing method.

The term “sensor information” refers to measurement data acquired from one or more sensing components associated with the user input/output device or an external device, such as motion sensors, biological sensors, or environmental sensors, including activity information, vital signs, and contextual metadata.

The term “symptom information” refers to information indicating a physical or psychological condition experienced by the user, such as pain, discomfort, or functional limitation, expressed in natural language input or derived from sensor information.

The term “request information” refers to information indicating preferences, wishes, or constraints of the user regarding treatment, exercise, or support, such as desired therapy type, intensity level, schedule, or facility characteristics.

The term “emotion-related information” refers to information that reflects or is used to infer an emotional state of the user, including explicit verbal expressions of feelings and implicit signals obtainable from voice, text, facial expressions, or physiological reactions.

The term “time information” refers to temporal data such as timestamps or time intervals that specify when input information or sensor information was acquired or processed.

The term “location information” refers to spatial data indicating a position of the user or the user input/output device, such as geographic coordinates, region identifiers, or facility proximity indicators.

The term “communication network” refers to a wired or wireless data transmission infrastructure, including the Internet, local area networks, or mobile communication networks, through which data are exchanged between the server, the user input/output device, and external systems.

The term “natural language processing method” refers to a computational technique for analyzing and transforming natural language input information, including processes such as tokenization, morphological analysis, part-of-speech tagging, entity recognition, and phrase extraction.

The term “morphological analysis” refers to a process that segments natural language text into smaller units such as morphemes or tokens and determines grammatical or lexical attributes of each unit.

The term “phrase extraction” refers to a process that identifies and selects meaningful word sequences, such as noun phrases or verb phrases, from natural language text in order to represent salient concepts such as symptoms or user requests.

The term “emotion analysis method” refers to a computational technique for estimating an emotion state of a user from data such as voice signals, textual content, facial images, or sensor readings, using rule-based analysis, statistical models, or machine learning models.

The term “emotion state” refers to a representation of a user's emotional condition, such as anxiety, fear, relief, or motivation, expressed as one or more categories, scores, or continuous values.

The term “activity information” refers to information indicating bodily movement or behavioral activity of the user, including metrics such as steps taken, exercise duration, intensity level, and frequency of sessions, obtained from a biological information recording device or other sensors.

The term “biological information recording device” refers to an apparatus that measures and records physiological or activity-related data of the user, such as a wearable device, a medical monitoring device, or an integrated sensor module.

The term “user state information” refers to a structured representation of the user's current condition generated by the processor, including at least symptom information, request information, emotion state, and activity information, and optionally further including medical record information and contextual metadata.

The term “medical record information storage device” refers to a storage subsystem or database system that holds clinical data associated with the user, such as diagnosis information, treatment history, test results, and outcome records.

The term “facility information storage device” refers to a storage subsystem or database system that holds information about treatment facilities or service providers, including attributes such as location, available services, specialties, and ratings.

The term “candidate treatment facilities” refers to one or more treatment facilities selected or ranked by the processor, based on user state information and stored facility data, as suitable options for providing treatment or support to the user.

The term “effectiveness indicators” refers to quantitative or qualitative measures that indicate a degree of success, benefit, or outcome of an existing or proposed treatment plan, such as predicted improvement scores, adherence metrics, or symptom-change indices.

The term “structured data” refers to information that has been organized into a defined schema or format, such as records with predetermined fields or arrays of key-value pairs, such that the information can be programmatically processed and queried by the processor.

The term “prompt sentence” refers to a text sequence generated by the processor that encodes instructions, context, and constraints for a generative AI model, and that is used as input to cause the generative AI model to generate corresponding output text.

The term “generative AI model” refers to an artificial intelligence model that generates output data, such as natural language text, from input data including prompt sentences, by performing learned transformations based on trained parameters.

The term “generated text” refers to a sequence of natural language characters or tokens output by the generative AI model in response to a prompt sentence, including treatment plan information, exercise plan information, explanations, or recommendations.

The term “treatment plan information” refers to information that specifies a proposed course of medical or therapeutic actions for a user, including types of treatments, frequencies, durations, and conditions for modification.

The term “exercise plan information” refers to information that specifies a proposed course of physical activities or exercises for a user, including exercise types, intensities, schedules, and instructions.

The term “explanatory information” refers to natural language content generated for the purpose of explaining aspects of a treatment plan, an exercise plan, or a recommendation to the user, including rationales, cautions, or educational descriptions.

The term “plan information” refers to a structured representation derived from generated text, including at least implementation items, precaution information, motivation messages, and candidate treatment facilities, which is suitable for storage, display, and further automated processing.

The term “implementation items” refers to discrete actionable elements contained in plan information, such as individual exercises, tasks, or scheduled activities associated with specific days or sessions.

The term “precaution information” refers to information within the plan that indicates warnings, restrictions, or conditions under which certain actions should be modified, postponed, or avoided for safety or appropriateness.

The term “motivation messages” refers to messages or phrases within the plan information intended to encourage, support, or positively influence the user's adherence and engagement with the plan.

The term “history information” refers to recorded data associated with a user over time, including previous plan versions, applied prompt sentences, execution records, feedback, and evaluation results stored in a recording device.

The term “execution status information” refers to information indicating whether and to what extent the user has performed the actions specified in the plan information, such as completion flags, timestamps, and measured activity values.

The term “feedback information” refers to information provided by the user regarding subjective experiences or evaluations related to the plan, such as perceived pain changes, difficulty levels, satisfaction, or suggestions.

The term “achievement indicators” refers to metrics computed by the processor that describe a degree of completion or adherence to the plan information by the user, including measures such as completion rates, session counts, and consistency over time.

The term “update prompt sentence” refers to a prompt sentence generated after initial plan generation, which includes at least achievement indicators, effectiveness indicators, or updated user state information, and which is input into the generative AI model to obtain revised or refined plan information.

The term “evaluation model” refers to a machine learning model executed by the processor to estimate or score treatment effectiveness or other outcome measures on the basis of activity information, medical record information, achievement indicators, or related features.

The term “treatment effectiveness” refers to an assessed or predicted impact of a treatment plan on a user's health condition, measured by objective or subjective indicators such as pain reduction, functional improvement, or risk decrease.

The term “emotion-related information” refers to data derived from or describing an emotion state estimation, including numerical scores, categorical labels, or descriptors such as anxiety level or motivation level.

The term “presentation contents” refers to a set of visual, textual, or auditory elements delivered to the user input/output device, including how plan information, explanations, and recommendations are arranged, emphasized, and formatted.

The term “selection criteria of the candidate treatment facilities” refers to rules, weights, or conditions used by the processor to select and rank candidate treatment facilities, based on factors such as medical suitability, distance, user preferences, and emotion-related information.

In one embodiment, a server cooperates with at least one terminal and at least one user to implement the system. The server includes one or more processors, a main memory, a persistent storage device, and a network interface. The server executes software components including an operating system, a web server, an application server, a database management system, a natural language processing library, a machine learning framework, and a client library for accessing an external generative AI model. The server may be implemented on general-purpose computing hardware such as a multi-core processor platform in a data center or a cloud-computing environment.

The terminal includes at least one processor, memory, a display device, an audio input/output device, one or more sensors, and a network interface. The terminal executes a client application implemented, for example, as a native application or a web application using a user interface framework. The terminal functions as a user input/output device in the sense of the claims.

The user interacts with the terminal to provide natural language input and to review and confirm plan information. The user may be an individual undergoing treatment, a caregiver, or a medical professional using the system on behalf of a patient.

The server stores in the persistent storage device one or more databases managed by a database management system such as a relational database system. The databases include at least a user database, a medical record information database, a facility information database, a plan history database, and a feature database. The user database holds profile data including identifiers, demographic information, and configuration preferences. The medical record information database holds structured clinical data for each user, including diagnosis codes, historical treatments, outcome scores, and timestamps. The facility information database holds attributes of treatment facilities, such as geographic coordinates, available modalities, and classification tags. The plan history database holds past versions of plan information, the prompt sentences used to generate the plans, and corresponding evaluation results. The feature database stores analytical features and indicators derived from raw data.

The server executes a natural language processing module implemented using a library such as a statistical or neural natural language processing toolkit. The natural language processing module includes a tokenizer, a morphological analyzer, a part-of-speech tagger, and an entity extractor. The server configures the entity extractor to detect medical-related terms, such as anatomical locations, symptom names, severity modifiers, and therapy types, using a combination of vocabulary lists and neural sequence labeling models. The server maps synonyms to canonical terms using a controlled vocabulary table stored in the database.

The server executes an emotion analysis module. In one embodiment, the emotion analysis module communicates with an external emotion recognition service that receives voice or text segments and returns probability values for a fixed set of emotion categories such as “anxiety,” “fear,” “relief,” and “motivation.” In another embodiment, the emotion analysis module executes a local neural classifier that processes feature vectors extracted from audio or text. For audio input, the terminal extracts acoustic features such as Mel-frequency cepstral coefficients and pitch statistics and sends these to the server, and the server applies a feedforward neural network or recurrent neural network to output emotion scores. For text input, the server applies a transformer-based encoder to compute contextual embeddings and then applies a classification layer to output emotion probabilities. The server discretizes the probabilities into emotion states by applying threshold rules stored in configuration tables.

The server executes an activity integration module to process sensor information. The terminal collects sensor information from motion and physiological sensors, such as accelerometers, gyroscopes, heart-rate sensors, and location sensors. The terminal aggregates the raw readings into basic activity metrics, such as total steps, exercise duration, walking distance, and approximate intensity, and sends these metrics periodically to the server. The server receives the metrics, aligns them to time windows using timestamps, and aggregates them into higher-level features such as weekly activity totals, adherence to prescribed sessions, and distribution of intensity levels. The server stores these aggregates in the feature database.

The server executes a medical record integration module that periodically retrieves medical record information from external clinical information systems via standardized program interfaces. The server normalizes external coding schemes into internal codes using rule tables and mapping functions. The server associates each record with a user identifier and stores the normalized clinical events in the medical record information database. The medical record integration module produces features such as counts of prior treatments, time since last major intervention, and trends of outcome scores.

The server executes an evaluation model module implemented using a machine learning framework. In one embodiment, the evaluation model is a neural network configured as a multi-layer perceptron that receives as input a feature vector composed of activity information features, medical record features, symptom severity indicators, and previous plan parameters. The evaluation model outputs numerical indicators such as predicted probability of improvement and estimated pain reduction. The server trains the evaluation model offline using historical data stored in the databases. During training, the server initializes model parameters, defines a loss function such as mean squared error or cross-entropy, and updates the parameters using gradient-based optimization algorithms. The server may perform data augmentation by sub-sampling time windows or adding noise to features to improve generalization. The trained model is stored in persistent storage and loaded into memory at runtime for inference.

The server executes a prompt construction module that generates prompt sentences for a generative AI model. The prompt construction module assembles fields from user state information and structured data into a textual sequence according to a predefined template. The user state information includes symptom descriptions, request information, emotion state, and activity information. The structured data includes candidate treatment facilities and effectiveness indicators obtained from the evaluation model. The server specifies an ordering and labeling scheme for inserting these fields into the prompt sentence, so that the generative AI model receives a consistent context across calls. The server may include meta-information such as required output sections, constraints on intensity, and time horizon in the prompt sentence.

The server calls a generative AI model through an interface provided by a remote service or a local model-serving component. The generative AI model in one embodiment is a transformer-based language model having multiple attention layers, feedforward layers, and learned token embeddings. The server sends the prompt sentence to the generative AI model, along with configuration parameters such as decoding strategy and output length. The generative AI model converts the prompt sentence into token embeddings, applies self-attention and feedforward operations layer by layer, and outputs probability distributions over possible next tokens at each position. The model generates a sequence of tokens representing the generated text. The server receives the generated text and records the prompt-response pair in the plan history database.

The server performs post-processing of the generated text using a plan structuring module. The plan structuring module splits the generated text into sections corresponding to implementation items, precaution information, motivation messages, and optional facility recommendations. The server uses pattern rules and heading markers embedded in the generated text to identify these sections. In one embodiment, the server instructs the generative AI model via the prompt sentence to output specific heading labels in the generated text, and the plan structuring module searches for those labels and parses the subsequent lines into structured fields. The server converts the parsed structures into a machine-readable representation, such as a set of tables or objects, and stores them as plan information in the plan history database.

The terminal retrieves the plan information from the server and presents it to the user through a graphical user interface. The terminal displays implementation items organized by dates or sessions, draws visual indicators such as progress bars to show adherence, and displays motivation messages and precautions near corresponding items. The terminal displays candidate treatment facilities on a map or as a ranked list. When the user interacts with the interface to mark tasks as completed or to enter feedback such as perceived pain levels, the terminal packages this information as execution status information and feedback information and sends it to the server.

The server receives the execution status information and feedback information and updates the user state information. The server calculates achievement indicators such as completion ratios and counts of skipped sessions, and updates effectiveness indicators using the evaluation model module by feeding the new features into the trained model. The server then executes the prompt construction module again to generate an update prompt sentence that incorporates the achievement indicators, updated effectiveness indicators, and any changes in emotion state. The server calls the generative AI model with the update prompt sentence to obtain revised generated text that modifies part or all of the existing plan information. The server updates the plan history database with the revision and transmits the updated plan information to the terminal.

The server implements data structures and processing flows in a way that improves computation efficiency and technical performance. The user state information is stored as a normalized record with fixed fields for symptom categories, request categories, numeric severity values, emotion scores, and aggregated activity metrics. The server uses separate index structures on user identifiers and timestamps to enable efficient queries for the latest state. The server batches feature extraction and evaluation model inference operations for multiple users to utilize vectorized arithmetic and reduce overhead. The server also caches common pattern fragments used in prompt sentences and reuses them across calls, reducing string construction time and network payload size. The server reduces communication load by sending incremental updates or compressed structures to the terminal, instead of full plan data, when only small changes occur.

The system improves computer technology in several respects. By constructing user state information as a unified, structured representation that combines natural language, sensor information, clinical data, and emotion states, the server can apply machine learning models and generative AI in a way that avoids redundant parsing and data conversion at every step. This organization reduces total processor time and memory usage compared to systems that repeatedly transform unstructured data at each stage. By using an evaluation model to compute effectiveness indicators and feeding those indicators back into the prompt sentence, the server causes the generative AI model to operate within a closed feedback loop, which reduces the number of iterations needed to obtain a satisfactory plan and lowers the total number of generative calls. By structuring the generated text into plan information with explicit fields, the server can perform algorithmic computations over plans, such as adherence calculations and conflict checks, that are not possible when treating generated text as unstructured strings. This structuring enables efficient search and update algorithms on the plan history database and reduces the cost of later retrieval and analysis.

The system applies non-conventional processing rules for prompt construction and plan updating that differ from manual or naive automation approaches. The server orders elements in the prompt sentence according to their impact on generative behavior, placing high-weight indicators such as predicted effectiveness and extreme emotion states at positions that the model was empirically shown to attend to strongly during fine-tuning. The server enforces internal consistency by inserting explicit constraints such as “avoid high-impact exercises” or “assume beginner level” into the prompt sentence when certain clinical flags are present. These rules are determined by analysis of model behavior and are encoded as part of the application logic, creating a specialized interaction between the evaluation model and the generative AI model that a human operator would not follow in real time due to complexity. In one concrete example, the user enters through the terminal the natural language statement “Recently I have low back pain and I would like a training plan mainly focused on stretching.” The terminal sends the text to the server. The server performs natural language processing and recognizes the canonical symptom term “low back pain” and the request category “stretching-focused plan.” The server reads recent activity information such as number of running sessions from the feature database and identifies that the user has performed multiple running sessions without improvement. The server runs the evaluation model and obtains an indicator that the current plan is ineffective. The server also runs the emotion analysis module on the user's text or voice and finds a mild anxiety state. The server builds a prompt sentence as follows:

“User health information: chronic low back pain. User preference: a training plan mainly focused on stretching. Recent activity data: three running sessions per week, 30 minutes each, no stretching. Detected emotion: mild anxiety about pain. Current running-based plan is evaluated as ineffective. Please generate a one-week stretching-centered exercise plan to reduce low back pain, including warm-up, main stretching exercises, and cool-down, with clear daily schedules and low-impact intensity suitable for a beginner.”

The server sends this prompt sentence to the generative AI model, receives detailed generated text describing specific exercises and schedules, and structures the text into plan information. The terminal displays the plan information on a calendar view and allows the user to follow and report on the plan.

In another example, the user expresses emotional difficulty, such as “I am still very anxious about going to the clinic.” The terminal sends this statement to the server. The server updates the emotion state to high anxiety and uses the medical record information and facility information databases to find candidate facilities that offer counseling or quiet environments. The server constructs a prompt sentence as follows:

“The user has chronic low back pain and currently feels high anxiety about visiting medical facilities. The user prefers facilities with a calm environment and supportive communication. Based on this state, please propose characteristics of suitable clinics and recommend how to present the treatment plan in a reassuring manner, avoiding technical jargon and emphasizing safety.”

The server sends this prompt sentence to the generative AI model, receives generated text describing types of facilities and suggested wording for explanations, structures the results, and updates the plan information and facility recommendations. The terminal presents this updated information to the user with visual emphasis on comfort-related attributes.

The system can be extended in multiple alternative embodiments. In one variation, the generative AI model is hosted locally on the server, and the server manages allocation of computational resources such as graphical processing units for inference. In such a case, the server can adjust decoding parameters and sequence lengths dynamically based on device load and network conditions, further improving communication efficiency. In another variation, the evaluation model is implemented as a gradient-boosted decision tree ensemble instead of a neural network, using features computed by the same feature extraction pipeline. In another variation, the emotion analysis module is implemented separately for audio and text, with ensemble rules combining their outputs into a single emotion state.

The server can also apply different scheduling and caching strategies. For example, the server can schedule evaluation and updating operations at specific intervals or when thresholds are reached, such as when the adherence ratio drops below a certain value. The server can cache prompt sentences and generated text for similar user states to reduce repeated model inference. The server can maintain a shared representation of common plan segments and reuse those segments across users when appropriate, reducing storage needs and generation time.

By specifying concrete data structures, modules, and neural network models, and by defining the interaction between evaluation models, prompt construction, and generative AI inference, the system provides a technical improvement in how heterogeneous patient-related data are processed and used to generate adaptive plans. The improved structuring of data, efficient prompt generation, and closed-loop updating mechanism collectively lead to faster convergence to effective plans, reduced computational load, and more precise alignment of generated content with sensor and clinical signals, thereby improving the functioning of the computer system itself rather than merely automating human mental activities.

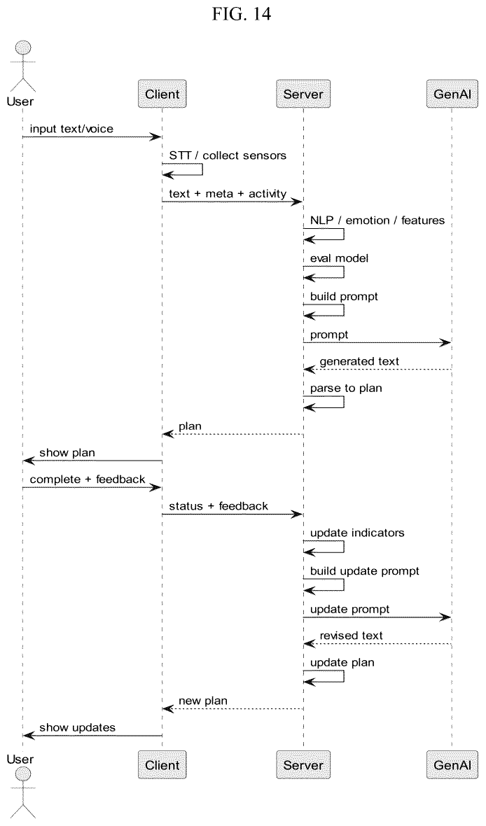

The following describes the processing flow using FIG. 14.

### Step 1:

User inputs symptom, request, and emotion-related information.

User enters natural language text such as “Recently I have low back pain and I would like a training plan mainly focused on stretching,” or speaks this content into the microphone of the terminal.

Input: Free-form natural language text or voice, optionally together with initial selections such as “exercise-based plan preferred.”

Output: Raw text string (for text input) or raw audio signal (for voice input) held in the memory of the terminal.

Terminal displays an input screen, records keystrokes into a text buffer for text input, or records audio samples from the microphone for voice input. Terminal stores the captured data with a temporary identifier and prepares it for further processing.

### Step 2:

Terminal converts voice to text and attaches metadata.

Terminal, when voice input is used, calls a speech recognition function to convert the raw audio signal into text. Terminal reads system time and location sensor values and binds these as metadata to the text.

Input: Raw audio signal and device context information (time, location, device ID).

Output: Natural language text string, timestamp, location coordinates, and device identifier packaged into a structured record.

Terminal segments the audio into frames, computes acoustic features, and sends them to a local or remote speech-to-text engine. Terminal receives recognized text, creates a data structure containing the text, time, and position, and stores it in local memory for transmission.

### Step 3:

Terminal collects local sensor and activity information.

Terminal reads motion and physiological sensors such as accelerometers and heart-rate sensors via platform APIs and aggregates raw readings into simple metrics.

Input: Raw sensor readings (acceleration, step count increments, heart-rate samples, GPS samples).

Output: Aggregated activity metrics such as daily step count, recent exercise duration, and approximate intensity values.

Terminal periodically samples the sensors, integrates step increments over defined intervals, averages heart-rate samples, computes distances from location changes, and writes the aggregated metrics into a local data structure associated with the user's current session.

### Step 4:

Terminal transmits input package to the server.

Terminal bundles natural language text, metadata, and aggregated activity metrics into a network message and sends it over a secure communication channel to the server.

Input: Structured record containing user text, timestamp, location, device identifier, and activity metrics.

Output: Network request containing the structured record delivered to the server's API endpoint.

Terminal serializes the record into a message format such as JSON, initiates an HTTPS request to a predefined server URL, attaches authentication information, and sends the serialized body. Terminal waits for an acknowledgment from the server.

### Step 5:

Server receives and parses user input.

Server listens on an API endpoint and receives the network request, then parses the serialized content into internal objects.

Input: Network message containing serialized user input record and activity metrics.

Output: In-memory representation of user text, metadata, and activity metrics separated into typed fields.

Server reads the request body, invokes a deserialization function, and maps the contents into internal variables such as “raw_text,” “timestamp,” “location,” and “activity_vector.” Server stores a copy of the raw record in a log table.

### Step 6:

Server performs natural language processing and keyword extraction.

Server applies a natural language processing library to the raw text to break it into tokens and identify medically relevant expressions.

Input: Raw text string from the user.

Output: Token list, part-of-speech tags, detected symptom terms, detected request terms, and a normalized set of canonical keywords.

Server invokes a tokenizer to split the text into tokens, passes tokens through a morphological analyzer and part-of-speech tagger, and applies a medical entity recognizer that labels spans corresponding to symptoms, body parts, and treatment types. Server consults a synonym mapping table to map different surface forms to canonical labels such as “low_back_pain” and “stretching_preference,” and stores these canonical keywords in a structured feature record.

### Step 7:

Server performs emotion analysis.

Server determines an emotion state associated with the user's input by processing either the text or voice-derived features.

Input: Raw text string, and optionally audio-derived features or text-derived embeddings.

Output: Emotion scores for multiple categories and a selected discrete emotion state label. Server forwards the text or acoustic features to an emotion classifier or an external emotion service, receives probability values for categories such as anxiety or fear, and stores these probabilities. Server compares each probability with thresholds defined in configuration data and selects a dominant category as the emotion state, for example “mild_anxiety.” Server adds the emotion scores and label to the current user state record.

### Step 8:

Server aggregates activity information and clinical history.

Server reads the activity metrics included in the request and merges them with previously stored metrics and clinical records.

Input: New activity metrics from the terminal and existing medical record entries from the medical record database.

Output: Aggregated features such as total runs per week, total exercise duration, time since last clinical visit, and recent outcome trends.

Server retrieves prior activity entries for the user based on the timestamp, adds current counts to weekly totals, and computes averages and trends. Server queries the medical record database for recent visits, treatments, and outcome scores, and generates summary values such as “three running sessions per week, no stretching” and “no significant improvement over two weeks.” Server writes these summary values into the feature database.

### Step 9:

Server constructs unified user state information.

Server combines natural language-derived keywords, emotion state, activity features, and clinical summaries into a single user state record.

Input: Canonical symptom and request keywords, emotion label and scores, activity summaries, and clinical summaries.

Output: User state information object containing structured fields that represent the current condition of the user.

Server creates a record with fields such as “symptom_category,” “request_category,” “emotion_state,” “weekly_activity_vector,” and “clinical_trend_vector.” Server assigns values from previous steps to these fields and stores the record in the user state table keyed by user identifier and timestamp.

### Step 10:

Server evaluates current or prior plan effectiveness.

Server uses an evaluation model to compute effectiveness indicators of any existing plan for the user, based on the user state information.

Input: User state information, parameters of the evaluation model, and metadata of the current or last plan.

Output: Effectiveness indicators such as predicted probability of improvement, estimated pain change, and a plan status flag (effective or ineffective).

Server loads the evaluation model into memory if not already loaded, converts the user state record and plan metadata into a numeric feature vector, and feeds the vector to the model's inference function. Server receives numerical outputs, interprets them as effectiveness values, and compares them with predefined thresholds to assign a qualitative plan status. Server stores the indicators and status in the feature database.

### Step 11:

Server retrieves candidate treatment facilities.

Server searches the facility information database for facilities that match the user's needs and context.

Input: Symptom and request categories, user location, and optional emotion state and plan status.

Output: List of candidate treatment facilities and associated attributes ranked by relevance. Server formulates a query that filters facilities by specialty tags matching “low_back_pain” and “exercise_therapy,” orders results by geographic distance from the user's location, and optionally boosts scores for facilities tagged as “calm_environment” when the emotion state is anxious. Server computes a composite relevance score for each facility, sorts the list, and packs the top results into a structured collection for later use.

### Step 12:

Server builds a prompt sentence for the generative AI model.

Server assembles a textual prompt that summarizes the user state, plan effectiveness, and facility context and defines clear instructions for the generative AI model.

Input: User state information, effectiveness indicators, and candidate facility summaries.

Output: Prompt sentence text string suitable for input to the generative AI model.

Server inserts symptom description, requests, activity summary, emotion state, and effectiveness assessment into a predefined textual template. For example, the server generates:

“User health information: chronic low back pain. User preference: a training plan mainly focused on stretching. Recent activity data: three running sessions per week, 30 minutes each, no stretching. Detected emotion: mild anxiety about pain. Current running-based plan is evaluated as ineffective. Please generate a one-week stretching-centered exercise plan to reduce low back pain, including warm-up, main stretching exercises, and cool-down, with clear daily schedules and low-impact intensity suitable for a beginner.”

Server stores the prompt text along with a reference to the user state record in the plan history database.

### Step 13:

Server invokes the generative AI model.

Server sends the prompt sentence to a generative AI model and obtains generated text.

Input: Prompt sentence string and model configuration parameters such as output length and decoding temperature.

Output: Generated text string containing proposed treatment or exercise plan details and explanatory information.

Server creates a request for the generative AI model containing the prompt and parameters, sends it over a secure channel to a model-serving endpoint, and waits for a response. The generative AI model processes the prompt by applying its internal transformer layers to produce a probability distribution over output tokens and then samples tokens to form a response. Server receives the full generated text and stores the prompt-response pair in the plan history database for traceability.

### Step 14:

Server structures the generated text into plan information.

Server parses the generated text and converts it into structured plan information.

Input: Generated text string from the generative AI model.

Output: Plan information object containing implementation items, precaution information, motivation messages, and any embedded facility recommendations.

Server scans the generated text for section headers or patterns such as “Day 1,” “Precautions,” and “Motivation,” splits the text into segments, and extracts individual tasks and instructions. Server assigns each task a date or session index, attaches associated precautions and motivational phrases, and converts each task into a structured record with fields such as “exercise_name,” “duration,” “intensity,” and “notes.” Server aggregates these records into a plan information object and writes it to the plan history database.

### Step 15:

Server transmits plan information to the terminal.

Server prepares a response containing the structured plan information and sends it back to the terminal.

Input: Plan information object and candidate facility list.

Output: Network response including plan structure and facility data.

Server serializes the plan information and facility list into a compact message, sets appropriate headers, and sends the response message via the communication network to the terminal's callback endpoint. Server logs the time and size of the transmission and links the response to the corresponding user state and prompt.

### Step 16:

Terminal renders the plan and collects execution data.

Terminal receives the plan information and displays it to the user in an interactive format, then starts monitoring execution.

Input: Structured plan information and facility list from the server.

Output: User-visible plan view and in-memory tracking structures for execution status.

Terminal parses the response message, populates a calendar or list view with daily tasks, displays facility names and locations, and highlights precautions. Terminal creates local records for each task to track completion status. As the user interacts with the interface and performs exercises, the terminal records completion times, subjective ratings (such as pain level after a session), and updated sensor metrics, forming new execution status records that can later be transmitted back to the server.

### Step 17:

User executes the plan and provides feedback.

User follows the displayed plan, performs the recommended exercises or actions, and reports experiences through the terminal.

Input: Visual and textual instructions presented on the terminal.

Output: User actions in the real world and feedback text or selections submitted to the terminal.

User reads daily tasks, starts exercises as instructed, and uses the interface to mark tasks as done, rate difficulty, or comment with statements such as “pain has eased slightly” or “these stretches are too intense.” User's input is captured by the terminal as new feedback information and status flags.

### Step 18:

Terminal sends execution status and feedback to the server.

Terminal collects completed-task records, feedback, and additional sensor information and transmits them to the server.

Input: Execution status records, user feedback text or ratings, and updated sensor metrics.

Output: Network request containing a summary of performed activities and user evaluations delivered to the server.

Terminal aggregates completion flags, timestamps, and ratings into structured entries, ties them to the original plan item identifiers, and packs them into a message. Terminal sends the message to the server using a secure channel, then resets local temporary buffers while maintaining local copies for offline viewing.

### Step 19:

Server updates indicators and user state and prepares an update prompt.

Server recalculates achievement and effectiveness indicators and generates an update prompt sentence for the generative AI model.

Input: New execution status information, feedback information, and current plan information.

Output: Updated achievement indicators, updated effectiveness indicators, revised user state information, and an update prompt sentence.

Server computes per-task completion ratios and overall adherence, updates summary features such as “adherence below 60%” or “pain decreased after week one,” and runs the evaluation model again with these updated features. Server refreshes the user state record with new emotion estimates derived from feedback text and recomputed activity summaries. Based on these values, server constructs a new prompt sentence, for example:

“User with chronic low back pain followed a stretching-centered exercise plan for one week with 60% adherence. The user reports mild pain reduction but still experiences stiffness in the morning. Detected emotion: moderate motivation, mild concern. Please revise the plan to slightly reduce intensity on busy days, add specific morning stretches, and include clear, encouraging messages to maintain adherence.”

Server stores the update prompt and updated indicators in the plan history database.

### Step 20:

Server refines the plan via the generative AI model.

Server sends the update prompt sentence to the generative AI model and obtains a refined plan, then updates the stored plan and notifies the terminal.

Input: Update prompt sentence and current plan information.

Output: Revised generated text and an updated plan information object.

Server invokes the generative AI model with the update prompt, receives new generated text that modifies or adds exercises and messages, and parses the text through the same structuring logic as before. Server computes the difference between the existing plan and the new plan, updates corresponding entries in the plan history database, and flags changed tasks. Server then transmits the updated plan information to the terminal so that the terminal can show the modifications to the user and continue the iterative process.

The data generation model 58 is a so-called generative artificial intelligence (AI). Examples of the data generation model 58 include generative Als such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Moreover, although the processing by the data processing system 10 described above was executed by the specific processing unit 290 of the data processing device 12 or by the control unit 46A of the smart device 14, the processing may be executed by a specific processing unit 290 of the data processing device 12 and a control unit 46A of the smart device 14. Moreover, the specific processing unit 290 of the data processing device 12 acquires and collects information needed for processing from the smart device 14 or from an external device or the like, and the smart device 14 acquires and collects information needed for processing from the data processing device 12 or from an external device or the like.

For example, a collection unit is implemented by the control unit 46A of the smart device 14 and/or by the specific processing unit 290 of the data processing device 12. For example, an acquisition unit acquires number-of-steps data using the camera 42 and/or the communication I/F 44 of the smart device 14, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the output device 40 of the smart device 14 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart device 14.

### Second Exemplary Embodiment

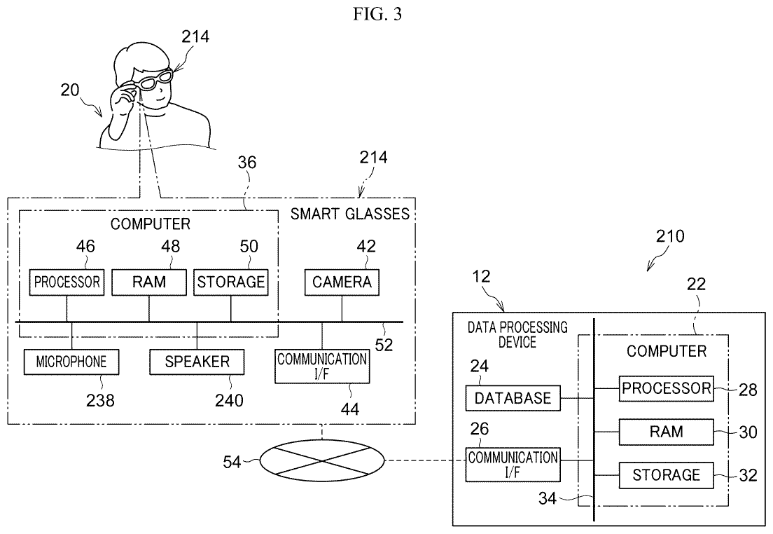

FIG. 3 illustrates an example of a configuration of a data processing system 210 according to a second exemplary embodiment.

As illustrated in FIG. 3, the data processing system 210 includes a data processing device 12 and smart glasses 214. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart glasses 214 include a computer 36, a microphone 238, a speaker 240, a camera 42, and a communication I/F 44. The computer 36 includes a processor 46, RAM 48, and storage 50. The processor 46, the RAM 48, and the storage 50 are connected to a bus 52. The microphone 238, the speaker 240, the camera 42, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

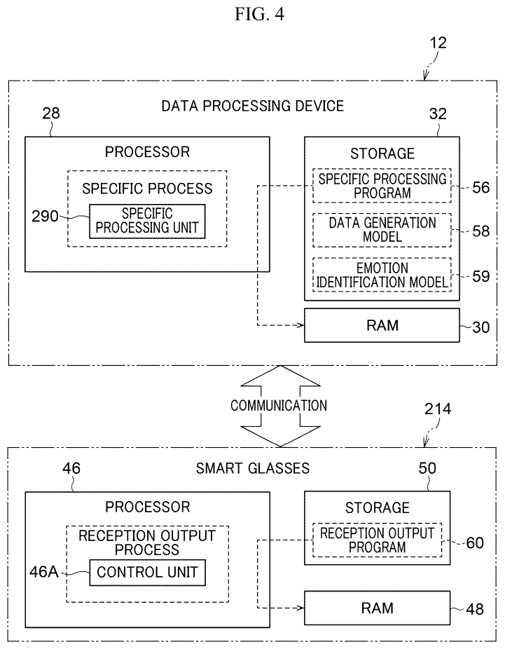

FIG. 4 illustrates an example of relevant functions of the data processing device 12 and the smart glasses 214. As illustrated in FIG. 4, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart glasses 214. A reception and output program 60 is stored in the storage 50. The processor 46 reads the reception and output program 60 from the storage 50 and in the RAM 48 executes the read reception and output program 60. The reception and output processing is implemented by the processor 46 operating as the control unit 46A according to the reception and output program 60 executed in the RAM 48. Note that a configuration may be adopted in which the smart glasses 214 include a data generation model and an emotion identification model similar to the data generation model 58 and the emotion identification model 59, and processing similar to the specific processing unit 290 is performed using these models.

Next, description follows regarding the specific processing by the specific processing unit 290 of the data processing device 12. The units of the system described below are implemented by the data processing device 12 and the smart glasses 214. In the following description the data processing device 12 is called a “server”, and the smart glasses 214 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the smart glasses 214. The control unit 46A in the smart glasses 214 outputs the specific processing result to the speaker 240. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the smart glasses 214, the processing may be executed by a specific processing unit 290 of the data processing device 12 and a control unit 46A of the smart glasses 214. Moreover, the specific processing unit 290 of the data processing device 12 acquires and collects information needed for processing from the smart glasses 214 or from an external device or the like, and the smart glasses 214 acquires and collects information needed for processing from the data processing device 12 or from an external device or the like.

For example, the collection unit is implemented by the control unit 46A of the smart glasses 214 and/or by the specific processing unit 290 of the data processing device 12. For example, an acquisition unit acquires number-of-steps data using the camera 42 and/or the communication I/F 44 of the smart glasses 214, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 of the smart glasses 214 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart glasses 214.

### Third Exemplary Embodiment

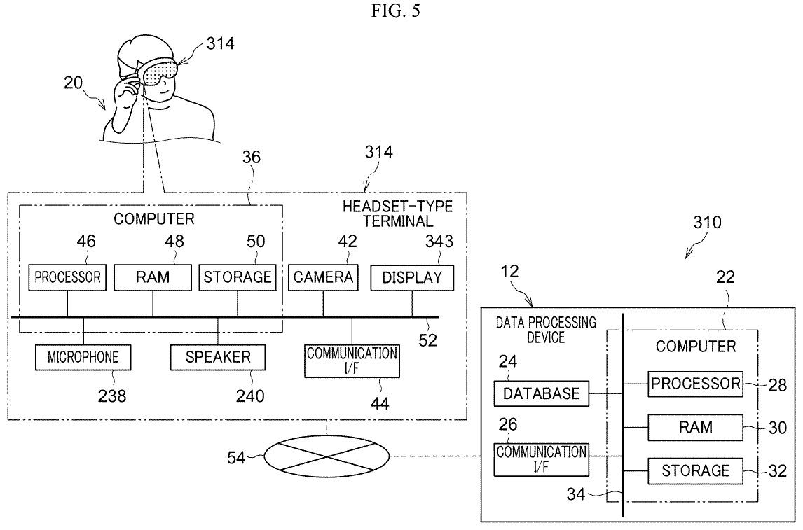

FIG. 5 illustrates an example of a configuration of a data processing system 310 according to a third exemplary embodiment.

As illustrated in FIG. 5, the data processing system 310 includes a data processing device 12 and a headset-type terminal 314. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The headset-type terminal 314 includes a computer 36, a microphone 238, a speaker 240, a camera 42, a communication I/F 44, and a display 343. The computer 36 includes a processor 46, RAM 48, and storage 50. The processor 46, the RAM 48, and the storage 50 are connected to a bus 52. The microphone 238, the speaker 240, the camera 42, the display 343, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

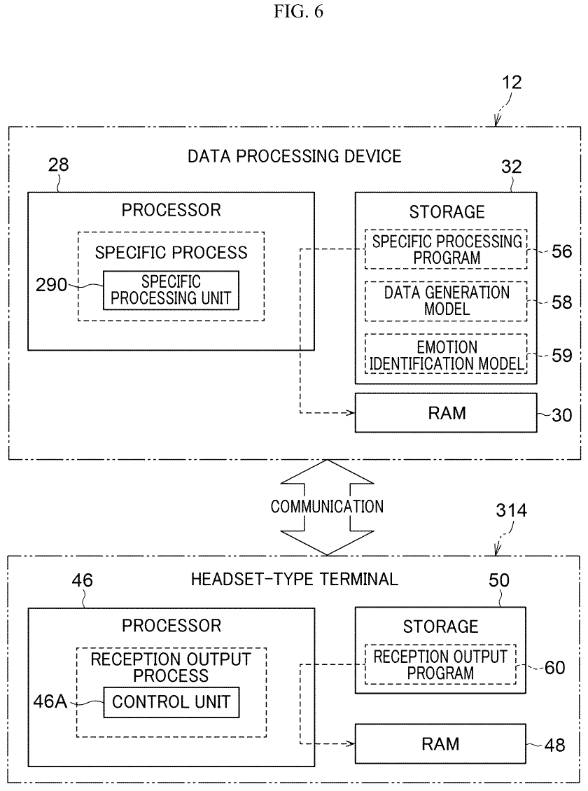

FIG. 6 illustrates an example of relevant functions of the data processing device 12 and the headset-type terminal 314. As illustrated in FIG. 6, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the headset-type terminal 314. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the headset-type terminal 314.

In the following description the data processing device 12 is called a “server”, and the headset-type terminal 314 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the headset-type terminal 314. In the headset-type terminal 314, the control unit 46A outputs the result of the specific processing to the speaker 240 and the display 343. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the headset-type terminal 314, the processing may be executed by a specific processing unit 290 of the data processing device 12 and a control unit 46A of the headset-type terminal 314. Moreover, the specific processing unit 290 of the data processing device 12 acquires and collects information needed for processing from the headset-type terminal 314 or from an external device or the like, and the headset-type terminal 314 acquires and collects information needed for processing from the data processing device 12 or from an external device or the like.

For example, the collection unit is implemented by the control unit 46A of the headset-type terminal 314 and/or by the specific processing unit 290 of the data processing device 12. For example, an acquisition unit acquires number-of-steps data using the camera 42 and/or the communication I/F 44 of the headset-type terminal 314, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the display 343 of the headset-type terminal 314 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the headset-type terminal 314.

### Fourth Exemplary Embodiment

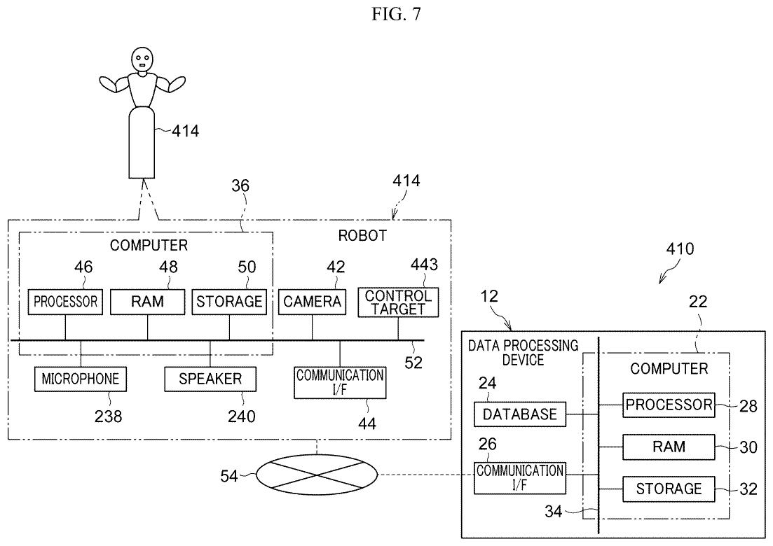

FIG. 7 illustrates an example of a configuration of a data processing system 410 according to a fourth exemplary embodiment

As illustrated in FIG. 7, the data processing system 410 includes a data processing device 12 and a robot 414. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The robot 414 includes a computer 36, a microphone 238, a speaker 240, a camera 42, a communication I/F 44, and a control target 443. The computer 36 includes a processor 46, RAM 48, and storage 50. The processor 46, the RAM 48, and the storage 50 are connected to a bus 52. The microphone 238, the speaker 240, the camera 42, the control target 443, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the robot 414 (for example, with an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

The control target 443 includes a display device, eye LEDs, and motors to drive arms, hands, feet, and the like. The posture and gesture of the robot 414 are controlled by controlling the motors of the arms, hands, feet, and the like. Part of an emotion of the robot 414 can be expressed by controlling these motors. Moreover, a facial expression of the robot 414 can be represented by controlling an illumination state of the eye LEDs of the robot 414.

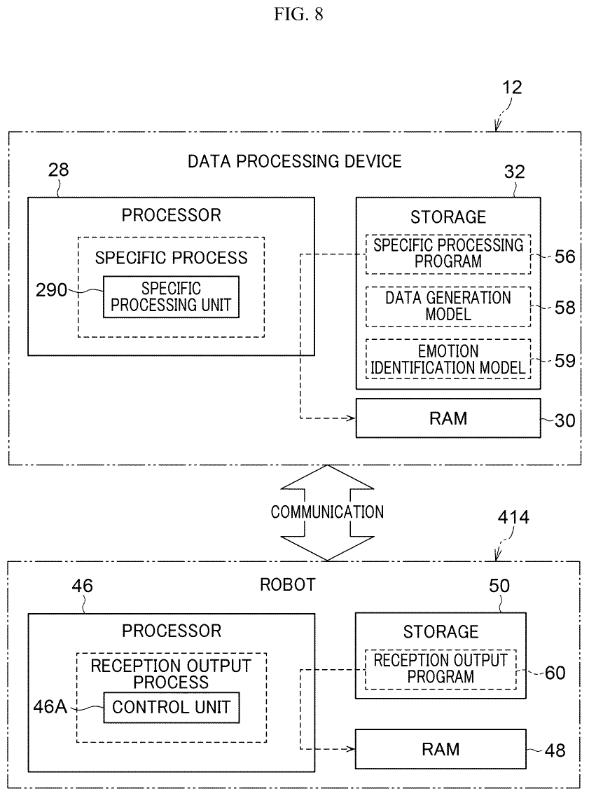

FIG. 8 illustrates an example of relevant functions of the data processing device 12 and the robot 414. As illustrated in FIG. 8, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the robot 414. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the robot 414. In the following description the data processing device 12 is called a “server”, and the robot 414 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the robot 414. In the robot 414, the control unit 46A outputs the result of the specific processing to the speaker 240 and the control target 443. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative Als such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the robot 414, the processing may be executed by a specific processing unit 290 of the data processing device 12 and a control unit 46A of the robot 414. Moreover, the specific processing unit 290 of the data processing device 12 acquires and collects information needed for processing from the robot 414 or from an external device or the like, and the robot 414 acquires and collects information needed for processing from the data processing device 12 or from an external device or the like.

For example, the collection unit is implemented by the control unit 46A of the robot 414 and/or by the specific processing unit 290 of the data processing device 12. For example, an acquisition unit acquires number-of-steps data using the camera 42 and/or the communication I/F 44 of the robot 414, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the control target 443 of the robot 414 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the robot 414.

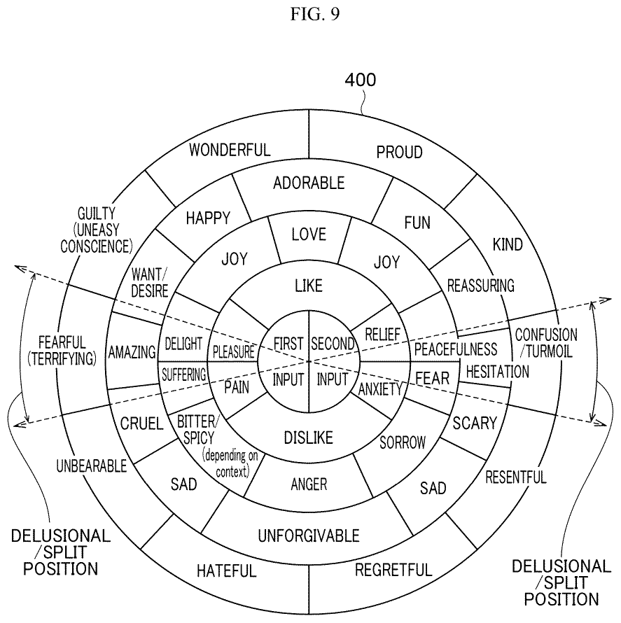

Note that the emotion identification model 59 serves as an emotion engine, and may decide the emotion of a user according to a specific mapping. Specifically, the emotion identification model 59 may decide the emotion of a user according to an emotion map (see FIG. 9) that is a specific mapping. Moreover, the emotion identification model 59 may also decide the emotion of the robot similarly, and the specific processing unit 290 may be configured so as to perform the specific processing using the emotion of the robot.

FIG. 9 is a diagram illustrating an emotion map 400 mapping plural emotions. In the emotion map 400, emotions are arranged in concentric circles that radiate out from the center. Primitive states of emotion are arranged nearer to the center of the concentric circles. Emotions expressing states and actions generated from states of mind are arranged further toward the outside of the concentric circles. Emotions are defined as including both affect and mental states. Emotions generated from reactions occurring in the brain are generally arranged at the left side of the concentric circles. Emotions induced by situational assessment are generally arranged at the right side of the concentric circles. Emotions generated from reactions occurring in the brain that are also emotions induced by situational assessment are generally arranged toward the top and toward the bottom of the concentric circles. Moreover, emotions of “euphoria” are arranged at the upper side of the concentric circles, and emotions of “dysphoria” are arranged at the lower side of the concentric circles. Plural emotions are accordingly mapped in this manner in the emotion map 400 based on a structure giving rise to emotions, and emotions that readily occur at the same time are mapped close to each other.

An example of such emotions is a distribution of emotions in the direction of 3 o'clock on the emotion map 400, generally around a boundary between relief and anxiety. Situational awareness dominates over internal sensations in the right half of the emotion map 400, with an impression of calm.

The inside of the emotion map 400 represents feelings, and the outside of the emotion map 400 represents actions, and so emotions further toward the outside of the emotion map 400 are more visible (are expressed by actions).

Human emotions are based on various balances, such as posture and blood sugar value balances, with a state of dysphoria being exhibited when these balances are far from ideal and a state of euphoria being exhibited when these balances are near to ideal. Even in a robot, a car, a motorbike, or the like, emotions can be thought of as being based on various balances such as orientation and remaining battery balances, with a state called dysphoria being exhibited when these balances are far from ideal and a state called euphoria being exhibited when these balances are near to ideal. An emotion map may, for example, be generated based on the emotion map of Dr. Mitsuyoshi (PhD Dissertation https://ci.nii.ac.jp/naid/500000375379: “Research on the phonetic recognition of feelings and a system for emotional physiological brain signal analysis”, Tokushima University). Emotions belonging to an area called “reaction” where feeling dominates are arranged in the left half of the emotion map. Moreover, emotions belonging to an area called “situation” where situational awareness dominates are arranged in the right half of the emotion map.

There are two types of emotion that facilitate leaning in an emotion map. One is an emotion in the vicinity of the center of negative “penitence” and “reflection” on the situational side. In other words, sometimes a negative “emotion” such as “I don't want to feel this way ever again” and “I don't want to be chided again” is experienced in a robot. Another is a positive emotion in the area of “desire” on the reaction side. In other words, there are times when a positive feeling such as “desire more” and “want to know more” is experienced.

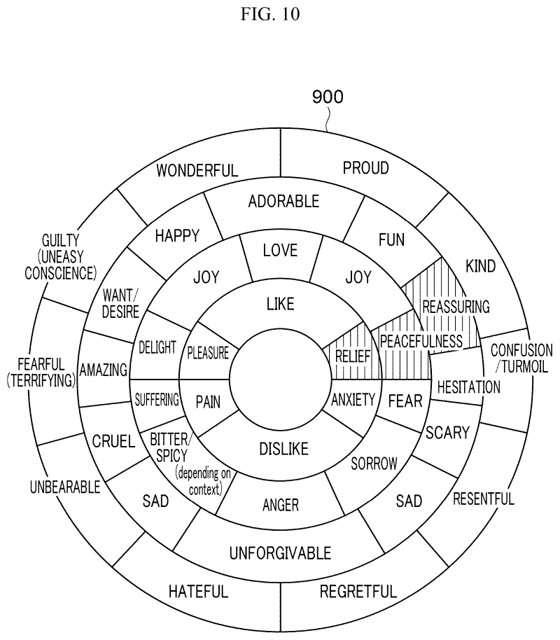

In the emotion identification model 59, user input is input to a pre-trained neural network, and emotion values indicating emotions shown on the emotion map 400 are acquired and the emotions of the user are decided. This neural network is pre-trained based on plural training data sets that each combine a user input with an emotion value indicating an emotion shown on the emotion map 400. The neural network is also trained such that emotions arranged close to each other have values that are close to each other, as in an emotion map 900 illustrated in FIG. 10. In FIG. 10 the plural emotions of “relief”, “peaceful”, and “reassured” are indicated as an example of close emotion values.

Although the system according to the present disclosure has been described mainly as functions of the data processing device 12, the system according to the present disclosure is not limited to being implemented in a server. The system according to the present disclosure may be implemented as a general information processing system. The present disclosure may, for example, be implemented by a software program operating on a personal computer, and may be implemented by an application operating on a smartphone or the like. The method according to the present disclosure may also be supplied to a user in the form of Software as a Service (SaaS).

Although in the exemplary embodiments described above examples are given of embodiments in which the specific processing is performed by a single computer 22, technology disclosed herein is not limited thereto, and distributed processing may be performed for the specific processing, with the specific processing distributed across plural computers including the computer 22. For example, the data generation model 58 may be provided in a device external to the data processing device 12, such that data generation in response to input data is performed in the external device.

Although in the exemplary embodiments described above examples are described of embodiments in which the specific processing program 56 is stored in the storage 32, the technology disclosed herein is not limited thereto. For example, the specific processing program 56 may be stored on a portable, non-transitory, computer readable, storage medium, such as universal serial bus (USB) memory or the like. The specific processing program 56 stored on the non-transitory storage medium is then installed on the computer 22 of the data processing device 12. The processor 28 then executes the specific processing according to the specific processing program 56.

Moreover, the specific processing program 56 may be stored on a storage device, such as a server connected to the data processing device 12 over the network 54, with the specific processing program 56 then being downloaded in response to a request from the data processing device 12 and installed on the computer 22.

Note that there is no need to store the entire specific processing program 56 on the storage device, such as a server connected to the data processing device 12 over the network 54, or to store the entire specific processing program 56 on the storage 32, and part of the specific processing program 56 may be stored thereon.

Hardware resources for executing the specific processing may use various processors as listed below. Examples of processors include, for example, a CPU that is a general-purpose processor that functions as a hardware resource to execute the specific processing by executing software, namely a program. Moreover, the processor may, for example, be a dedicated electronic circuit that is a processor having a circuit configuration custom designed for executing the specific processing, such as a field-programmable gate array (FPGA), a programmable logic device (PLD), or an application specific integrated circuit (ASIC). Memory is inbuilt or connected to each of these processors, and the specific processing is executed by each of these processors using the memory.

The hardware resource that executes the specific processing may be configured from one of these various processors, or may be configured from a combination of two or more processors of the same or different type (for example, a combination of plural FPGAs, or a combination of a CPU and a FPGA). The hardware resource executing the specific processing may be a single processor.

Examples of configurations of a single processor include, firstly, a configuration of a single processor resulting from combining one or more CPU and software, in an embodiment in which this processor functions as the hardware resource for executing the specific processing. Secondly, as typified by a System-on-chip (SOC) or the like, there is also an embodiment that uses a processor realized by a single IC chip to function as an overall system including plural hardware resources for executing the specific processing. Adopting such an approach means that the specific processing is realized using one or more of the various processors described above as hardware resource.

Furthermore, more specifically, an electrical circuit that combines circuit elements such as semiconductor elements or the like may be employed as a hardware structure of these various processors. The specific processing is merely an example thereof. This means that obviously redundant steps may be omitted, new steps may be added, and the processing sequence may be swapped around within a range not departing from the spirit of the present disclosure.

The described content and drawing content illustrated above are a detailed description of parts according to the present disclosure, and are merely examples of the present disclosure. For example, description related to the above configuration, function, operation, and advantageous effects is a description related to examples of the configuration, function, operation, and advantageous effects of parts according to the present disclosure. This means that obviously redundant parts may be eliminated, new elements may be added, and switching around may be performed on the described content and drawing content illustrated above within a range not departing from the spirit of the present disclosure. Moreover, to avoid misunderstanding and to facilitate understanding of parts according to the present disclosure, description related to common knowledge in the art and the like not particularly needing description to enable implementation of the present disclosure is omitted in the described content and drawing content illustrated as described above.

All publications, patent applications and technical standards mentioned in the present specification are incorporated by reference in the present specification to the same extent as if each individual publication, patent application, or technical standard was specifically and individually indicated to be incorporated by reference.

Note that, regarding the above description, the following supplementary notes are further disclosed.

### Example 1

### (Supplementary 1)

A system comprising a processor,
wherein the processor is configured to
provide an information input and output interface through which a user inputs physical condition information and preference condition information,
obtain natural language data transmitted from a use terminal and extract, by language analysis processing including tokenization, part-of-speech tagging, and important term extraction, abstract matters related to symptoms and abstract matters related to preference conditions from the natural language data,
set a search condition for an information storage device that stores healthcare service point information based on the extracted abstract matters, and execute a search process including structured query processing and full-text search to acquire candidate healthcare service point information,
generate a prompt sentence to be input to a generative information processing apparatus based on the extracted abstract matters and the acquired healthcare service point information, input the prompt sentence to the generative information processing apparatus and obtain, from the generative information processing apparatus, generated information including a selection result of healthcare service points suitable for the user's symptoms and preference conditions and explanatory information related to the selection result,
compare and associate the acquired healthcare service point information with the generated information, and generate integrated healthcare service point information in which ranking, filtering, and addition of explanatory information of the healthcare service points are performed based on the generated information, and
transmit the integrated healthcare service point information to the use terminal and provide output data in a displayable format to the user.

### (Supplementary 2)

The system according to supplementary 1,
wherein the processor is configured to
configure the prompt sentence, based on the user's physical condition information and preference condition information and on the candidate healthcare service point information, as content that instructs automatic generation of an individualized treatment plan including time-series treatment content and lifestyle guidance content for the user, obtain, from the generative information processing apparatus, generated information including the individualized treatment plan, and associate the individualized treatment plan with the integrated healthcare service point information and include the individualized treatment plan in the output data.

### (Supplementary 3)

The system according to supplementary 1,
wherein the processor is configured to
include, as additional input information in the prompt sentence, evaluation information and progress information from the user regarding the individualized treatment plan, configure the prompt sentence as content that instructs the generative information processing apparatus to generate an improvement proposal for the individualized treatment plan based on the evaluation information and the progress information, update the individualized treatment plan based on the improvement proposal obtained from the generative information processing apparatus, and provide, again as the output data, the updated individualized treatment plan together with the integrated healthcare service point information.

### Application Example 1

### (Supplementary 1)

A system comprising a processor,
wherein the processor is configured to
provide, on an information input/output device, an input screen through which a user inputs symptom information and request information,
acquire position information from the information input/output device, and temporarily store the acquired position information in association with the symptom information and the request information as acquisition data,
analyze natural language included in the acquisition data by using a natural language processing method, extract a term relating to a symptom and a term relating to a treatment, and generate a prompt sentence including an extraction result and input the prompt sentence into a generative information processing model,
analyze output information obtained from the generative information processing model, convert attribute information of a medical service provider included in the output information into internal classification information, and determine facility classification information, search medical facility information stored in an information storage device on the basis of the facility classification information and the position information, and extract a set of candidate medical facilities that satisfy a reliability condition and a distance condition,
rank the set of candidate medical facilities on the basis of priority information based on the output of the generative information processing model, evaluation information, and distance information, and generate search result information as structured data,
associate the search result information with map information corresponding to the position information, transmit display data to the information input/output device, and cause the information input/output device to perform map display and list display of the candidate medical facilities, and
receive reservation information, for a selected medical facility among the candidate medical facilities, from the information input/output device, generate a reservation record on the basis of the reservation information, and notify the selected medical facility of the reservation record.

### (Supplementary 2)

The system according to supplementary 1,
wherein the processor is configured to
generate the prompt sentence by including, in addition to the symptom information and the request information described in natural language, a constraint condition that instructs the generative information processing model to output the facility classification information and attribute information corresponding to the facility classification information in a structured format having predetermined keys, and obtain machine-readable structured output from the generative information processing model.

### (Supplementary 3)

The system according to supplementary 1,
wherein the processor is configured to
dynamically set a search condition for medical facilities by using priority information and recommended attribute information included in the output information obtained from the generative information processing model, and search the medical facility information while updating a search range and an evaluation criterion on the basis of the search condition and the position information.

### Example 2

### (Supplementary 1)

A system comprising a processor,
wherein the processor is configured to
provide, to a terminal, an input screen on an information processing apparatus for allowing a user to input symptom information and treatment request information of a patient,
receive, from the terminal via a secure communication scheme, the symptom information and the treatment request information, store the symptom information and the treatment request information as structured data, and generate and record identification information corresponding to the patient based on the structured data,
perform preprocessing on the structured data, the preprocessing including character string normalization processing and mandatory item verification processing, select a prompt sentence template based on a result of the preprocessing, the prompt sentence template being configured to receive the symptom information and the treatment request information, and generate a patient-specific prompt sentence by inserting the structured data into predetermined positions in the prompt sentence template,
add constraint condition information relating to treatment content and instruction information relating to an output format of a generation result to the patient-specific prompt sentence to construct an input prompt sentence for a generative AI model, and generate request data including the input prompt sentence and generation parameters,
transmit, via a network, a processing request to an external generative AI model using the request data, and acquire response data including treatment plan information generated by the generative AI model in response to the input prompt sentence,
extract the treatment plan information from the response data, perform formatting processing on the treatment plan information, the formatting processing including removal of leading and trailing whitespace, conversion of line break codes, and division of the treatment plan information into logical sections including at least a summary section, an exercise therapy section, and a lifestyle guidance section, and store formatted treatment plan information in association with the identification information, and
generate display data including the formatted treatment plan information and transmit the display data to the terminal so as to cause a display device of the terminal to display, for each patient, a customized treatment plan based on the formatted treatment plan information.

### (Supplementary 2)

The system according to supplementary 1,
wherein the processor is configured to
receive, from the terminal, update information including additional condition information or constraint condition information relating to the patient, record the update information in association with the structured data and the identification information, reconstruct the patient-specific prompt sentence based on the structured data and the update information, generate an updated input prompt sentence reflecting the additional condition information or the constraint condition information, input the updated input prompt sentence to the generative AI model again, acquire a revised version of the treatment plan information from the generative AI model, and transmit the revised version of the treatment plan information to the terminal.

### (Supplementary 3)

The system according to supplementary 1,
wherein the processor is configured to
receive, from the terminal, feedback information from the patient or a medical worker regarding the treatment plan information, store the feedback information in association with the identification information and the treatment plan information, generate an input prompt sentence for instructing the generative AI model to improve the treatment plan based on history information including the feedback information, input the input prompt sentence to the generative AI model, and acquire, for improvement of a treatment success degree of the patient, treatment plan information that is continuously improved based on the history information.

### Application Example 2

### (Supplementary 1)

A system comprising a processor,
wherein the processor is configured to
acquire, via a user input/output device, natural language input information and sensor information including symptom information, request information, and emotion-related information of a user, append time information and location information to the acquired input information, and transmit and receive the input information through a communication network,
analyze the acquired natural language input information by using a natural language processing method to perform morphological analysis and phrase extraction, extract phrases corresponding to the symptom information and the request information, analyze emotion from voice information or text information by using an emotion analysis method, and
generate user state information including the symptom information, the request information, an emotion state, and activity information obtained from a biological information recording device,
search, on the basis of the user state information, data stored in a medical record information storage device and a facility information storage device, calculate candidate treatment facilities and effectiveness indicators of existing treatment plans, and generate structured data including the candidate treatment facilities and the effectiveness indicators,
generate, on the basis of the user state information and the structured data, a prompt sentence for causing a generative AI model to generate a response different for each user, input the prompt sentence into the generative AI model, and obtain generated text including treatment plan information or exercise plan information and explanatory information,
analyze the generated text to structure the generated text as plan information including implementation items on a daily basis or on a session basis, precaution information, motivation messages, and the candidate treatment facilities, store the structured plan information as history information for each user in a recording device, and transmit the structured plan information to the user input/output device, and
calculate achievement indicators and effectiveness indicators of the plan information on the basis of execution status information, feedback information, and additional emotion information acquired from the user input/output device, generate an update prompt sentence reflecting the indicators, and input the update prompt sentence into the generative AI model again to continuously update the plan information.

### (Supplementary 2)

The system according to supplementary 1,
wherein the processor is configured to execute an evaluation model based on a machine learning method on the basis of the activity information, the medical record information, and the achievement indicators, obtain an evaluation result of treatment effectiveness, include the evaluation result in the prompt sentence, and input the prompt sentence into the generative AI model to dynamically adjust contents of the treatment plan information or the exercise plan information.

### (Supplementary 3)

The system according to supplementary 1,
wherein the processor is configured to estimate a degree of anxiety or a motivation level of the user on the basis of the emotion state and the activity information, generate emotion-related information including the estimation result, add the emotion-related information to the prompt sentence, and input the prompt sentence into the generative AI model to change presentation contents of the treatment plan information or the exercise plan information and selection criteria of the candidate treatment facilities in accordance with the emotion state of the user.

What is claimed is:

1. A system comprising:
circuitry configured to:
receive, via a communication interface coupled to a packet-switched network, natural language input data from a terminal device of a user;
perform language analysis processing on the natural language input data, the language analysis processing including tokenization and term extraction, to extract a first set of descriptor terms and a second set of preference terms from the natural language input data;
set a search condition for a data store based on the extracted first set of descriptor terms and the extracted second set of preference terms, and execute a search operation on the data store to acquire a set of candidate information records;
generate a prompt sentence based on the extracted descriptor terms, the extracted preference terms, and the acquired set of candidate information records, and transmit the prompt sentence to a generative language model via the communication interface;
receive, from the generative language model, generated information including a selection result and explanatory information corresponding to the selection result;
integrate the acquired set of candidate information records with the generated information to produce ranked output data in which the candidate information records are ordered and annotated based on the generated information; and
transmit the ranked output data as one or more data packets to the terminal device via the communication interface for display rendering.

2. The system according to claim 1, wherein the circuitry is configured to packetize the ranked output data into a plurality of transport-layer data packets formatted according to a network protocol for transmission over the packet-switched network to a registered network address of the terminal device.

3. The system according to claim 1, wherein the circuitry is configured to generate an extended prompt sentence that instructs the generative language model to produce an individualized action plan including time-series action content and guidance content for the user based on the descriptor terms and the preference terms, receive the individualized action plan from the generative language model, and associate the individualized action plan with the ranked output data for inclusion in the transmitted data packets.

4. The system according to claim 3, wherein the circuitry is configured to receive, from the terminal device, evaluation data and progress data from the user regarding the individualized action plan, include the evaluation data and the progress data as additional input in a subsequent prompt sentence, transmit the subsequent prompt sentence to the generative language model, receive an improvement proposal from the generative language model, and update the individualized action plan based on the improvement proposal.

5. The system according to claim 4, wherein the circuitry is configured to iteratively repeat the updating of the individualized action plan across a plurality of feedback cycles, storing each version of the individualized action plan in association with corresponding evaluation data in a storage area.

6. The system according to claim 1, wherein the language analysis processing further includes part-of-speech tagging and important term weighting, and wherein the circuitry is configured to assign relevance scores to the extracted descriptor terms based on frequency and positional information within the natural language input data.

7. The system according to claim 1, wherein the circuitry is configured to acquire position information from the terminal device, store the position information in association with the natural language input data, and constrain the search operation to candidate information records satisfying a distance condition relative to the acquired position information.

8. The system according to claim 7, wherein the circuitry is configured to rank the set of candidate information records based on a combination of priority information derived from the generated information, evaluation score data stored in the data store, and distance values computed from the position information, and generate the ranked output data as structured data.

9. The system according to claim 7, wherein the circuitry is configured to associate the ranked output data with map data corresponding to the position information, generate display data including the map data and a list representation of the candidate information records, and transmit the display data as a notification data packet to the terminal device.

10. The system according to claim 1, wherein the circuitry is configured to receive a reservation request for a selected candidate information record from the terminal device, generate a reservation record based on the reservation request, store the reservation record in the data store, and transmit a confirmation data packet to the terminal device via the communication interface.

11. The system according to claim 1, wherein the circuitry is configured to select a prompt template from a plurality of stored prompt templates based on a preprocessing result of the natural language input data, insert the extracted descriptor terms and preference terms into predetermined positions in the selected prompt template to generate the prompt sentence.

12. The system according to claim 11, wherein the circuitry is configured to add output-format constraint information to the prompt sentence, the output-format constraint information instructing the generative language model to produce the generated information in a structured format having predetermined keys.

13. The system according to claim 1, wherein the circuitry is configured to extract the generated information from a response received from the generative language model, perform formatting processing on the generated information including dividing the generated information into logical sections, and store formatted output in association with a user identifier in the storage area.

14. The system according to claim 1, wherein the circuitry is configured to apply an emotion identification model to at least one of audio data and text data received from the terminal device to estimate an emotional state of the user, and include the estimated emotional state in the prompt sentence.

15. The system according to claim 14, wherein the emotion identification model maps input data to emotion values according to an emotion map in which a plurality of emotions are arranged based on a structure giving rise to each emotion, and wherein emotions that co-occur are mapped to proximate positions in the emotion map.

16. The system according to claim 1, wherein the circuitry is configured to acquire sensor data from a measurement device associated with the user via the communication interface, derive activity information and physiological indicators from the sensor data, and include the activity information and the physiological indicators in the prompt sentence.

17. The system according to claim 1, wherein the circuitry is configured to encode the ranked output data with a display controller command that causes the terminal device to render the candidate information records as selectable interface elements, each selectable interface element linked to associated explanatory information from the generated information.

18. A system comprising:
circuitry configured to:
receive, via a communication interface coupled to a packet-switched network, structured input data from a terminal device, the structured input data including condition descriptor data and request data of a user;
store the structured input data in a storage area in association with an identification record for the user;
perform preprocessing on the structured input data including character normalization and mandatory item verification, select a prompt template based on a result of the preprocessing, and generate a user-specific prompt sentence by inserting the structured input data into the selected prompt template;
add constraint information and output-format instruction information to the user-specific prompt sentence to construct an input prompt sentence, and transmit the input prompt sentence as one or more data packets to a generative language model via the communication interface;
receive response data from the generative language model, extract action plan information from the response data, perform formatting processing on the action plan information including dividing the action plan information into a plurality of logical sections, and store formatted action plan information in the storage area in association with the identification record;
transmit display data including the formatted action plan information to the terminal device via the communication interface; and
receive, from the terminal device, feedback data from the user regarding the formatted action plan information, store the feedback data in the storage area, generate an updated prompt sentence incorporating the feedback data, transmit the updated prompt sentence to the generative language model, and receive revised action plan information for continuous improvement of the action plan.

19. The system according to claim 18, wherein the circuitry is configured to packetize the display data into one or more transport-layer data packets for transmission to a registered network address of the terminal device, and wherein the plurality of logical sections include at least a summary section, an activity section, and a guidance section.

20. A method performed by circuitry, the method comprising:
receiving, via a communication interface coupled to a packet-switched network, natural language input data from a terminal device of a user;
performing language analysis processing on the natural language input data to extract descriptor terms and preference terms;
setting a search condition based on the extracted terms and executing a search operation on a data store to acquire candidate information records;
generating a prompt sentence based on the extracted terms and the candidate information records, and transmitting the prompt sentence to a generative language model;
receiving generated information from the generative language model and integrating the candidate information records with the generated information to produce ranked output data; and
transmitting the ranked output data as one or more data packets to the terminal device via the communication interface.
